(19) United States
(12) Patent Application Publication
(10) Pub. No.: US 2008/0039935 A1
(43) Pub. Date: Feb. 14, 2008

(54) METHODS AND APPARATUS FOR MITRAL VALVE REPAIR

(76) Inventors: Wally BUCH, Atherton, CA (US); Fidel F. REALYVASQUEZ, Palo Cedro, CA (US)

Correspondence Address: LEVINE BAGADE HAN LLP, 2483 EAST BAYSHORE ROAD, SUITE 100, PALO ALTO, CA, 94303, US

(21) Appl. No.: 11/838,111

(22) Filed: Aug. 13, 2007

Related U.S. Application Data
(60) Provisional application No. 60/822,360, filed on Aug. 14, 2006.

(51) Int. Cl. A61F 2/24

(52) U.S. Cl. 623/2.17; 623/2.38

(57) ABSTRACT

Methods and apparatus for mitral valve repair are disclosed herein where the posterior mitral leaflet is supported or buttressed in a frozen or immobile position to facilitate the proper coaptation of the leaflets. An implantable apparatus may be advanced and positioned intravascularly beneath the posterior leaflet of the mitral valve. The apparatus may include one or more individual balloon members, each of which may be optionally configured with supporting integrated structures. A magnet chain catheter may be positioned within the coronary sinus and adjacent to the mitral valve to magnetically secure the apparatus in position beneath the posterior mitral leaflet. Alternatively, a split-ring device may be placed about the chordae tendineae supporting the mitral valve such that the ring slides along the chordae tendineae alternately against the mitral leaflet and towards the papillary muscles during systole and diastole.

### CROSS-REFERENCE TO RELATED APPLICATIONS

The application claims the benefit of priority to U.S. Prov. Pat. App. 60/822,360 filed Aug. 14, 2006, which is incorporated herein by reference in its entirety.

### FIELD OF THE INVENTION

The invention relates to methods and apparatus for valve repair in a patient body. More particularly, the invention relates to methods and apparatus for mitral valve repair for correcting conditions such as mitral valve regurgitation.

### BACKGROUND OF THE INVENTION

Essential to normal heart function are four heart valves, which allow blood to pass through the four chambers of the heart in a specified direction. These valves have either two or three cusps or leaflets, which are comprised of fibrous tissue that are attached to the walls of the heart. The cusps open when the blood is flowing correctly and then close to form a tight seal to prevent backflow.

The four chambers are known as the right and left atria (upper chambers) and right and left ventricle (lower chambers). The four valves that control blood flow are known as the tricuspid, mitral, pulmonary and aortic valves. In a normal functioning heart, the tricuspid valve allows inflow of deoxygenated blood from the right upper chamber (right atrium) to the right lower chamber (right ventricle). When the right ventricle contracts, the pulmonary valve allows one-way outflow from the right ventricle to the pulmonary vascular bed which carries deoxygenated blood to the lungs. The tricuspid valve is closed during this time. The mitral valve, also a one-way inflow valve, allows oxygenated blood which has returned to the left upper chamber (left atrium) to flow to the lower left chamber (left ventricle). When the left ventricle contracts, the oxygenated blood is pumped through the aortic valve to the aorta. During left ventricular ejection of blood, the mitral valve is closed. When the ventricle is at the end of its contractile state, the aortic valve begins to close and the cardiac cycle repeats itself.

Clinical cardiac decomposition (or heart failure) results from heart valve malfunction, such as mitral insufficiency. Mitral valve insufficiency, also known as mitral regurgitation, is a common cardiac abnormality where the mitral valve leaflets do not completely close when the left ventricle contracts. This allows blood to backflow into the left atrium resulting in left ventricular overload and if the condition is not corrected, the added workload will eventually cause left ventricular enlargement and dysfunction resulting in heart failure.

Various approaches to remedy mitral valve pathology typically require open heart surgery and have included various treatments such as valve replacement, chordae tendineae shortening or replacement, leaflet resection and mitral annular repair also known as annuloplasty. Annuloplasty and valvuloplasty procedures have been developed to correct mitral valve insufficiency.

Mitral valve insufficiency typically results from ischemia of the papillary muscles (chronic ischemic mitral regurgitation or CIMR) or connective tissue degeneration of the mitral leaflets or chordae tendineae. A combination of these factors can coexist in the same patient. Mitral regurgitation can also result from a change in the size and shape of the mitral annulus. For instance, the posterior annulus may enlarge to a greater degree than the anterior annulus. This is generally because the anterior annulus is attached to the strong fibrous skeleton of the heart while the posterior annulus is supported by cardiac muscle (a much more elastic tissue).

Procedures such as annuloplasty for achieving competence of the regurgitant mitral valve frequently require placement of a mitral annuloplasty ring. Studies have shown that ring annuloplasty abolishes dynamic annular motion and immobilizes the posterior leaflet. Rings of various designs used to perform annuloplasty can have an adverse effect on mitral valve function. For instance, where mitral valve repair with a prosthetic annuloplasty ring has been performed, reduced posterior leaflet motion is typically observed echocardiographically after most ring annuloplasty procedures.

Such reduced posterior leaflet functioning has been demonstrated to occur universally and is identical with either a semi-rigid or flexible complete annuloplasty ring. It is accepted that the ring stabilizes the posterior annulus and reinforces the posterior leaflet as this stabilization and reinforcement of the posterior leaflet is believed to create a buttress against which the anterior leaflet closes.

However, the “freezing” of the posterior leaflet effectively creates a uni-leaflet valve from a bi-leaflet valve. The clinical acceptance of posterior leaflet immobilization after mitral valve annuloplasty is felt to negatively impact the distribution of closing stress on the leaflets. The potential downside is increased collagen deposition resulting in leaflet thickening which can further stress leaflet closure.

Accordingly, a percutaneous mitral valve annuloplasty system that can effectively repair conditions such as mitral valve regurgitation without the drawbacks described above is desired.

### SUMMARY OF THE INVENTION

Supporting the posterior leaflet in a frozen or immobile position may not only alleviate stress imparted upon both leaflets but also enable both posterior and anterior leaflets to properly coapt in use, particularly for alleviating conditions such as mitral valve regurgitation. As such, an implantable device may be advanced and positioned intravascularly beneath the posterior leaflet of the mitral valve utilizing any number of percutaneous techniques.

One method of intravascularly treating the mitral valve may include advancing a guiding catheter having a distal end with a magnetic tip into a patient and through the vasculature and into the right atrium where the catheter may be articulated to enter the ostium of the coronary sinus. Once within the coronary sinus, the catheter may be advanced until a distal portion of the catheter is adjacently positioned relative to the posterior mitral leaflet of the mitral valve. With the guiding catheter positioned within the coronary sinus, a separate delivery catheter may also be introduced percutaneously into the patient and advanced into the patient's heart. The delivery catheter may be advanced into the patient's left ventricle through any number of approaches.

With delivery catheter desirably positioned within the left ventricle along, against, and/or adjacent to the inferior surface of the posterior mitral leaflet, a balloon catheter assembly may be advanced along the delivery catheter until the assembly is aligned along the posterior mitral leaflet proximate of the magnetic tip. The balloon catheter assembly may generally have one or more inflatable balloons which are aligned in series.

Each balloon may be interconnected to one another and each may define a lumen such that the balloon catheter assembly may be advanced over or along delivery catheter as an assembly. Moreover, each balloon may each have a corresponding inflation lumen through which one or more balloons may be inflated. Each balloon may be sized to correspond with a particular anatomical portion of the posterior mitral leaflet such that the balloon assembly as a whole may align with at least a majority of the length of the posterior mitral leaflet. Moreover, each or all of the balloons may be inflated to varying degrees relative to one another.

Any number of fluids or gases may be utilized, e.g., saline, water, contrast material, carbon dioxide, etc. In additional variations, alternative materials such as polymers may be utilized to fill the balloons and in yet other variations, each of the balloons may additionally be constructed to expand without the need for an inflation fluid or gas. For instance, one or more balloons may utilize an expandable scaffolding or structure to provide for expansion of the members against the posterior mitral leaflet.

With the balloon catheter assembly in position against or along the posterior mitral leaflet, a magnet chain catheter may be advanced over the guiding catheter into the coronary sinus proximate to the balloon catheter assembly. Alternatively, a guidewire may be left within the coronary sinus while the guiding catheter is withdrawn proximally from the coronary sinus and the magnet chain catheter is advanced along the guidewire into the coronary sinus in place of the guiding catheter.

In either case, the magnet chain catheter may generally be comprised of one or more magnets linearly aligned along a length of the outer surface of the catheter and these magnets may have a polarity opposite to the magnets integrated within the balloon catheter assembly. As the magnet chain catheter is advanced into position within the coronary sinus, the one or more magnets may be magnetically drawn towards the magnets integrated, within the balloon catheter assembly such that when aligned relative to one another, the balloon catheter assembly may be held securely in position relative to the posterior mitral leaflet by the magnet chain catheter. The balloon catheter assembly removably connected to the catheter shaft may alternatively utilize a single inflation shaft having an adjustable occluding mechanism to inflate each balloon member independently of one another.

Alternative variations for the balloon membrane may include variations where one or more of the balloon membranes include an expandable scaffold integrated within or upon the balloon membrane as an expandable woven or braided structure. In yet another variation, the balloon members may have one or more expandable rings integrated within the balloons. In yet another variation, a balloon variation may have an integrated stent-like structure expandable from a low-profile delivery configuration to an expanded deployment configuration.

Yet another example of an alternative apparatus which may be utilized to support the mitral valve, particularly the posterior mitral leaflet, in a frozen or immobile position to facilitate the proper coaptation of the posterior and anterior mitral leaflets may include a device configured as a split ring having two terminal atraumatic ends in apposition to one other separated by a split. The ring may have a central opening defined by the partial circumferential shape of the ring and may further form an open channel which is defined around the length of the ring. The channel may be enclosed along a top side of the ring by a presentation surface.

In use, because the chordae tendineae may loosely pass through the central opening, the ring may freely slide in vivo along the chordae tendineae while retained by the atraumatic ends. During systole, because of the tissue contraction and forced blood flow, the ring may be urged to slide along the chordae tendineae into a superior position where presentation surface is urged or pressed against the posterior mitral leaflet. As the presentation surface is pressed against the mitral valve, the posterior mitral leaflet may be supported by the ring in inhibiting or preventing prolapse of the leaflet.

### BRIEF DESCRIPTION OF THE DRAWINGS

FIG. 1A illustrates a representative side view of a mitral valve with the anterior and posterior mitral leaflets coapted relative to one another.

FIG. 1B illustrates an implantable support or buttress positioned inferiorly to the posterior mitral leaflet to freeze or immobilize movement of the leaflet into its closed position.

FIG. 1C generally illustrates the anatomy of the mitral valve and the positioning of the anterior mitral leaflet and the posterior mitral leaflet.

FIG. 2 illustrates a guiding catheter having a magnetic tip advanced within the coronary sinus such that the distal portion of the catheter is adjacently positioned relative to the posterior mitral leaflet.

FIG. 3 illustrates a delivery catheter advanced into the left ventricle via the aortic valve.

FIG. 4 illustrates the positioning of the delivery catheter along the posterior mitral leaflet relative to the guiding catheter within the coronary sinus.

FIG. 5 illustrates a balloon catheter assembly advanced along the delivery catheter into position along the posterior mitral leaflet.

FIG. 6 illustrates a magnet chain catheter advanced along the guiding catheter into position relative to the balloon catheter assembly.

FIG. 7 illustrates the delivery catheter removed from the balloon catheter assembly.

FIG. 8 illustrates the inflation of the one or more balloon members against the posterior mitral leaflet of the balloon catheter assembly.

FIG. 9 shows one variation for positioning of the one or more magnets within the balloon catheter assembly.

FIGS. 10A and 10B show alternative perspective views of a delivery catheter advanced intravascularly having a singular inflatable balloon member for placement against the posterior mitral leaflet.

FIGS. 11A and 11B show alternative perspective views of an inflated balloon catheter assembly positioned along or against the inferior surface of the proximal mitral leaflet within the left ventricle and inferior to the left atrium of the patient heart.

FIG. 12 illustrates a perspective view of one variation of the system where the balloon assembly may be attached, coupled, or otherwise removably connected via a catheter coupling mechanism to a catheter outer shaft or tubing.

FIG. 13 illustrates a perspective view of another variation of the balloon catheter assembly which utilizes a single inflation shaft having an adjustable occluding mechanism to inflate each balloon member independently of one another.

FIGS. 14A and 14B illustrate exploded assembly and exploded cross-sectional assembly views, respectively, of the balloon assembly of FIG. 13.

FIG. 15A illustrates a perspective view showing an infusion catheter shaft having a helical rail illustratively positioned within a lumen of the balloon members.

FIG. 15B illustrates a perspective view showing an infusion catheter shaft rotatably disposed within the wall occluding shaft to illustrate the advancement and retraction mechanism.

FIG. 16 shows the wall occluding shaft translated within the inflation shaft with its first opening aligned with the first opening defined along the length of the inflation shaft.

FIGS. 17A and 17B show partial cross-sectional perspective and detail views, respectively, illustrating one method for inflating the first balloon member.

FIG. 18 shows a fluid or gas being passed through the infusion lumen such that the second balloon interior is filled accordingly.

FIG. 19 shows the wall occluding shaft rotated or moved longitudinally such that the opening defined in the shaft is aligned with the opening in inflation shaft such that the lumen of the infusion catheter is in communication with the third balloon interior while communication with the first and second balloon interiors is precluded.

FIGS. 20A to 20C illustrate perspective views of another variation of the balloon assembly having a hollow tubular channel passing through each of the members.

FIGS. 21A to 21C show perspective, side, and end views, respectively, of another variation where the balloon members are un-symmetrically shaped.

FIG. 22 illustrates another variation of the balloon assembly where one or more of the balloon membranes may include an expandable scaffold.

FIG. 23 illustrates another variation of the balloon assembly where one or more expandable rings may be positioned along one or both ends of each balloon member to provide integrity to the expanded configuration.

FIGS. 24A and 24B illustrate perspective and partial cross-sectional perspective views, respectively, of another balloon variation having an integrated stent-like structure.

FIGS. 25A and 25B illustrate perspective superior and inferior views, respectively, of the balloon variation of FIGS. 24A and 24B deployed against or along the inferior surface of the posterior mitral leaflet.

FIG. 26 illustrates perspective view of a variation of the balloon inflation assembly where each balloon member has its own respective expandable stent structure.

FIGS. 27A to 27C illustrate partial cross-sectional views of a first stent deployed within a first balloon member.

FIGS. 28A and 28B illustrate partial cross-sectional views of a second stent deployed within a second balloon member.

FIGS. 29A and 29B illustrate partial cross-sectional views of a third stent deployed within a third balloon member.

FIG. 30A illustrates one variation of a detachment mechanism for releasing the balloon assembly from the catheter shaft.

FIG. 30B illustrates the inflation or expansion of the release mechanism balloon such that it radially contacts the coupling mechanism stent and urges it into an outward radial direction to release the coupling stent from catheter outer shaft.

FIG. 31A illustrates deflation of the release mechanism balloon to allow the catheter shaft and the delivery catheter to be withdrawn from the balloon assembly.

FIG. 31B illustrates the complete withdrawal of the catheter shaft from the balloon assembly.

FIG. 32 illustrates a partial cross-sectional view of the balloon assembly having the delivery catheter removed.

FIG. 33 illustrates a perspective assembly view of the coupling mechanism released with the delivery catheter and the catheter shaft being removed from the balloon assembly.

FIG. 34 shows a perspective view of another variation of a single balloon member for clarity with an infusion lumen in communication with the balloon member.

FIGS. 35A and 35B illustrate partial cross-sectional views showing the balloon interior with a closed unidirectional valve and with a distal end of the lumen breaching the valve to inflate the balloon member, respectively.

FIG. 36 shows a perspective detail view of an inflation assembly coupled to a delivery catheter via a release mechanism where the inflatable balloon members are spherically shaped and individually inflated via a separate inflation lumen.

FIGS. 37A and 37B illustrate partial cross-sectional views of first and second balloon members positioned along the inflation shaft and illustrating the inflation ports within each member for inflation with a fluid.

FIGS. 38A to 38C show perspective and cross-sectional end views, respectively, of a configuration of the delivery catheter having the multiple inflation lumens in communication with the inflation assembly.

FIGS. 39A to 39H illustrate perspective views of another method for delivering and positioning an inflation assembly inferiorly to a posterior mitral leaflet of the mitral valve within a patient heart.

FIGS. 40A and 40B illustrate alternate perspective views of an alternative apparatus configured as a split ring having two terminal atraumatic ends.

FIG. 41 illustrates a partial cross-sectional view of the apparatus of FIGS. 40A and 40B situated within the left ventricle and inferior to the mitral valve such that the ring is placed at least partially around the chordae tendineae supporting the mitral leaflets.

FIGS. 42A and 42B illustrate movement of the apparatus of FIGS. 40A and 40B between a superior position supporting the mitral valve leaflets and an inferior position along the chordae tendineae during systole and diastole, respectively.

### DETAILED DESCRIPTION OF THE INVENTION

By supporting a portion of the mitral valve, particularly the posterior leaflet, in a frozen or immobile position while avoiding reduction of the mitral annulus, a buttress may be created against which the anterior leaflet may close. By maintaining the posterior mitral leaflet frozen or immobile in its closed position, this may alleviate stress imparted upon both leaflets and enable both posterior and anterior leaflets to properly coapt in use, particularly for alleviating conditions such as mitral valve regurgitation. Generally, an implantable device may be advanced and positioned intravascularly beneath the posterior leaflet of the mitral valve utilizing any number of percutaneous techniques.

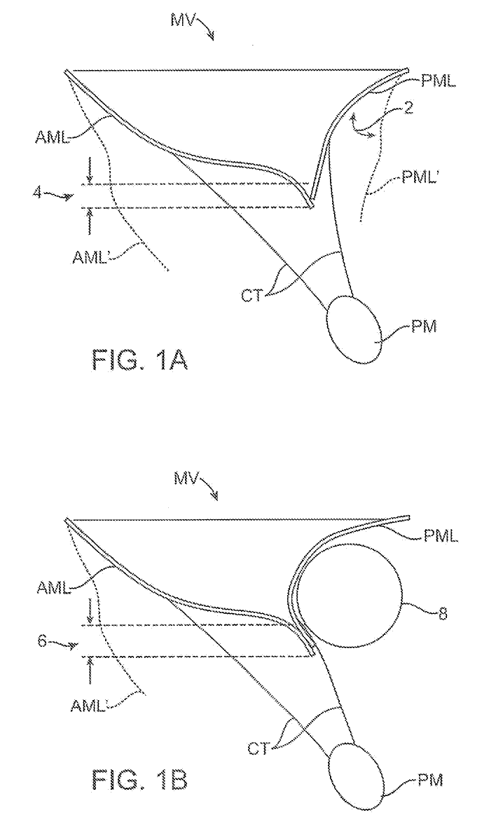

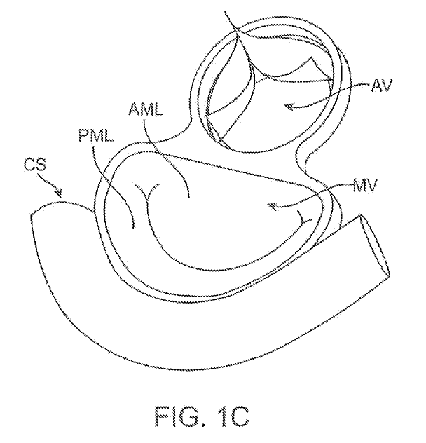

A representative side view of the anterior mitral leaflet AML and posterior mitral leaflet PML of a mitral valve MV are illustrated in FIG. 1A. When the heart is in systole, the leaflets are typically opposed relative to one another over a coaptation length 4, as shown. During diastole, the mitral valve MV opens where leaflets AML′, PML′ move away from one another. In particular, the posterior mitral leaflet PML may be seen moving through an excursion angle 2 from its closed position in apposition with the anterior mitral leaflet AML to its open position PML′. For a number of reasons, the leaflets AML, PML may fail to coapt relative to one another or their coaptation length 4 may be insufficient to provide proper sealing resulting in mitral regurgitation. For instance, one or more of the papillary muscles PM supporting the chordae tendineae CT, which are connected to the mitral leaflets, may become displaced or increases in transmitral pressure may increase annular tethering to interfere with proper leaflet coaptation.

As mentioned above, an implantable support or buttress 8 may be positioned inferiorly to the posterior mitral leaflet PML to freeze or immobile movement of the leaflet into its closed position while allowing the anterior mitral leaflet AML to move uninhibited between its closed and open configuration, as shown in FIG. 1B. Immobilizing the position of posterior mitral leaflet PML into its closed configuration may allow for the anterior mitral leaflet AML to properly close upon the posterior mitral leaflet PML and to maintain or increase its coaptation length 6.

FIG. 1C generally illustrates the anatomy of the mitral valve MV within a patient's heart showing the anterior mitral leaflet AML in apposition to the posterior mitral leaflet PML. A portion of the coronary sinus CS is shown passing generally adjacent to the mitral valve MV as well as the location of the aortic valve AV relative to the mitral valve MV.

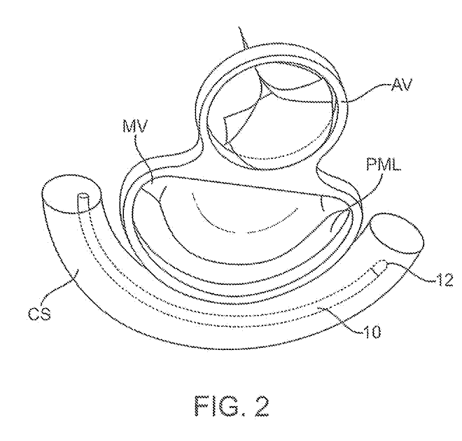

In one method of intravascularly treating the mitral valve, a guiding catheter 10 having a distal end with a magnetic tip 12 may be percutaneously inserted into a patient and advanced through the vasculature, e.g., via the inferior vena cava or the superior vena cava, and into the right atrium where the catheter 10 may be articulated to enter the ostium of the coronary sinus CS. Once within the coronary sinus CS, the catheter 10 may be advanced until a distal portion of the catheter is adjacently positioned relative to the posterior mitral leaflet PML of the mitral valve MV, as shown in FIG. 2.

The catheter 10 may comprise any number of configurations such as an articulatable catheter having a steerable tip to facilitate access within the vasculature. Moreover, the catheter 10 may be advanced optionally under the guidance of any number of visualization modalities, such as fluoroscopy, echocardiography, ultrasound, computed tomography (CT), magnetic resonance imaging (MRI), etc., if so desired. Additionally, catheter 10 may be advanced within the patient optionally under the guidance of a guidewire, as conventionally known. Although magnetic tip 12 may generally comprise a ferrous magnet, other variations of tip 12 may utilize an actuatable electromagnetic tip, in which case electrical wires may be routed through catheter 10 to activate the electromagnetic tip when desired or necessary.

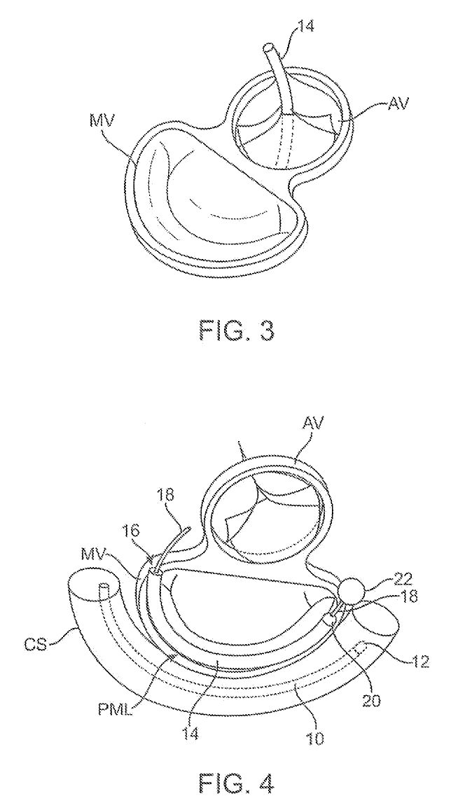

With the guiding catheter 10 positioned within the coronary sinus CS, a separate delivery catheter 14 may also be introduced percutaneously into the patient and advanced into the patient's heart. The delivery catheter 14 may be advanced into the patient's left ventricle through any number of approaches. For instance, in one variation, the delivery catheter 14 may be introduced and positioned within the heart via a retrograde arterial percutaneous access. Accordingly, the delivery catheter 14 may be introduced through a femoral access point and advanced through the abdominal aortic artery, through the aortic valve, and directly into the left ventricle, as shown in FIG. 3, where the catheter 14 may be articulated into position, as further described below. As above, delivery catheter 14 may be advanced and/or positioned under any number of imaging modalities as well as optionally under the guidance of a guidewire 18, as illustrated in FIG. 4.

Alternatively in another variation, the delivery catheter 14 may also be advanced through the inferior vena cava or superior vena cava and passed or otherwise pierced trans-septally through the atrial septum into the left atrium and articulated to enter directly through the mitral valve MV itself between the leaflets and into the left ventricle, where it may be articulated into position, as described below.

Once the distal portion of delivery catheter 14 has been advanced into the left ventricle, it may be steered or otherwise articulated such that it becomes positioned along, against, and/or adjacent to die inferior surface of the posterior mitral leaflet PML adjacent to guiding catheter 10 disposed within the coronary sinus CS, as further illustrated in FIG. 4. Delivery catheter 14 may also have a magnetic tip 20 configured to have a polarity opposite to magnetic tip 12 disposed on guiding catheter 10 such that as delivery catheter 14 is positioned along the posterior mitral leaflet PML, the magnetic attraction between each magnetic tip 12, 20 may facilitate the placement or positioning of delivery catheter 14 relative to the posterior mitral leaflet PML and guiding catheter 10.

Prior to, while, or even after delivery catheter 14 is desirably positioned, an inflatable or expandable member 22 positioned at a distal end of guidewire 18 may be actuated to inflate or expand against the surrounding tissue, such as any adjacent chordae tendineae, to hold or maintain a position of delivery catheter 14 relative to the posterior mitral leaflet. Guidewire 18 may be passed through a lumen 16 defined through delivery catheter 14. Where use of guidewire 18 is omitted, an inflatable or expandable member may be incorporated directly onto a portion of delivery catheter 14, for instance near or at a distal end of the catheter 14.

Although inflatable or expandable member 22 is illustrated as an inflatable balloon, which may be less likely to become lodged or entangled in any secondary chordae tendineae, other variations of expandable members may be utilized. For instance, other variations may utilize an expandable cage or scaffold made from a reconfigurable shape memory metal, such as a Nickel-Titanium alloy, or shape memory polymers.

Moreover, although introduction and positioning of guiding catheter 10 is illustrated as being prior to the introduction and positioning of delivery catheter 14, other variations may have both catheters 10, 14 introduced and advanced simultaneously into position within the heart. Yet other variations may alternatively include delivery catheter 14 introduced and positioned prior to placement of guiding catheter 10. Additional variations and alternatives may also be utilized as so desired and are intended to be included within the description herein.

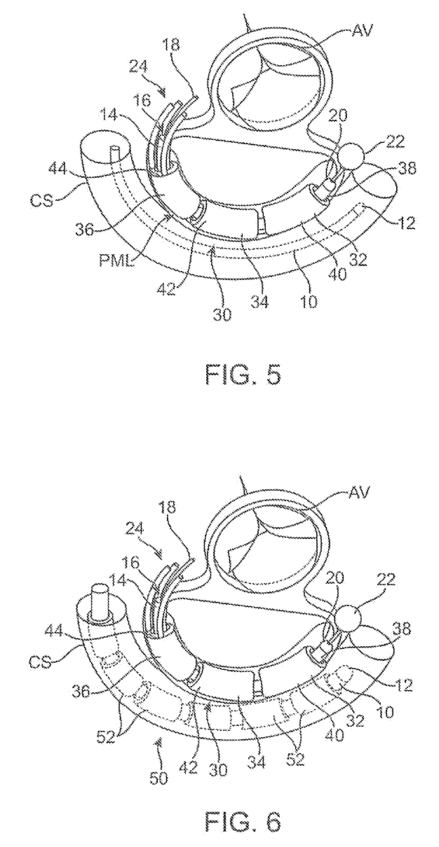

With delivery catheter 14 desirably positioned within the left ventricle along, against, and/or adjacent to the inferior surface of the posterior mitral leaflet PML, balloon catheter assembly 30 may be advanced along delivery catheter 14 until assembly 30 is aligned along the posterior mitral leaflet PML proximally of magnetic tip 20, as shown in FIG. 5. Balloon catheter assembly 30, in this particular variation which is described below in further detail, may generally have one or more inflatable balloons which are aligned in series.

As illustrated, assembly 30 may have first inflatable balloon 32 located distally, second inflatable balloon 34 located proximally of first balloon 32, and third inflatable balloon 36 located proximally of second balloon 34. Each balloon 32, 34, 36 may be interconnected to one another and each may define a lumen 38 such that balloon catheter assembly 30 may be advanced over or along delivery catheter 14 as an assembly. Moreover, each balloon 32, 34, 36 may each have a corresponding inflation lumen 24 through which one or more balloons may be inflated. Each balloon may be sized to correspond with a particular anatomical portion of the posterior mitral leaflet PML such that balloon assembly 30 as a whole may align with at least a majority of the length of the posterior mitral leaflet PML.

Balloon assembly 30 may be inflated in any combination, for instance, once securely positioned, all three balloon 32, 34, 36 may be uniformly inflated against the along the posterior mitral leaflet PML. Alternatively, any single one of the balloons 32, 34, 36 may be inflated alone without the remaining two balloons being inflated. In another alternative, any two of the balloons 32, 34, 36 may be inflated in combination without inflating the third balloon. For instance, first 32 and third balloons 36 may be inflated without inflating second balloon 34 or first 32 and second balloons 34 may be inflated without inflating third balloon 36, and so on in any number of combinations. Moreover, each or all of the balloons 32, 34, 36 may be inflated to varying degrees relative to one another. For instance, first balloon 32 may be fully inflated while the remaining balloons are partially inflated or not inflated at all. Alternatively, first balloon 32 may be partially inflated or not inflated at all, while second 34 and/or third balloons 36 are each or alternatively fully of partially inflated or not inflated at all.

The ability to alter the inflation and amount of inflation in each and/or all of the balloons 32, 34, 36 may allow for the practitioner to alter or customize the amount, degree, and/or positioning of buttressing provided along the length of the posterior mitral leaflet PML. This allows for a greater degree of flexibility in treating such leaflet deficiencies depending upon a particular patient's anatomy.

In other variations, although three inflation balloons are illustrated, alternative numbers of balloons may be utilized. For instance, a single balloon (further described below) for positioning relative to the posterior mitral leaflet PML may be utilized while in other variations, four or more balloons accordingly sized for placement against or along the posterior mitral leaflet PML may be utilized as practicable.

Moreover, in inflating the balloons, any number of fluids or gases may be utilized, e.g., saline, water, contrast material, carbon dioxide, etc. In additional variations, alternative materials such as polymers may be utilized to fill the balloons and in yet other variations, each of the balloons may additionally be constructed to expand without the need for an inflation fluid or gas. For instance, one or more balloons may utilize an expandable scaffolding or structure to provide for expansion of the members against the posterior mitral leaflet PML, as further described below.

In all these variations, these examples are intended to be illustrative and are not limiting. Accordingly, any and all combinations of the various features described above may be utilized with one another, e.g., combinations between varying inflation of the balloons, varying inflation amounts, number of balloons, inflation fluids or gases, expansion mechanisms, etc., are intended to be within the scope of this disclosure.

Aside from inflation or expansion of the one or more balloons 32, 34, 36, there may be one or more magnets integrated within the catheter or balloon assembly 30. For instance, a first magnet 40 may be integrated within or along first balloon 32, second magnet 42 may be integrated within or along second balloon 34, and third magnet 44 may be integrated within or along third balloon 36. These one or more magnets 40, 42, 44 may be simply integrated along the length of the assembly 30 and may alternatively utilize more than three magnets. Moreover, these magnets 40, 42, 44 may comprise ferrous magnets or alternatively utilize electromagnets.

Turning now to FIG. 6, with balloon catheter assembly 30 in position against or along the posterior mitral leaflet PML, magnet chain catheter 50 may be advanced over guiding catheter 10 into the coronary sinus CS proximate to the balloon catheter assembly 30. Alternatively, a guidewire may be left within the coronary sinus CS while guiding catheter 10 is withdrawn proximally from the coronary sinus CS and magnet chain catheter 50 is advanced along the guidewire into the coronary sinus CS in place of the guiding catheter 10.

In either case, magnet chain catheter 50 may generally be comprised of one or more magnets 52 linearly aligned along a length of the outer surface of the catheter 50 and these magnets 52 may have a polarity opposite to the magnets 40, 42, 44 integrated within balloon catheter assembly 30. The lengths of the one or more magnets 52 along catheter 50 may be sufficient to correspond at least with a length of the balloon catheter assembly 30, as illustrated. As magnet chain catheter 50 is advanced into position within the coronary sinus CS, the one or more magnets 52 may be magnetically drawn towards the magnets 40, 42, 44 integrated within balloon catheter assembly 30 such that when aligned relative to one another, balloon catheter assembly 30 may be held securely in position relative to the posterior mitral leaflet PML by magnet chain catheter 50. Accordingly, magnet chain catheter 50 acts as an anchoring element for balloon catheter assembly 30 utilizing magnetic attraction between the complementary magnetic attractor elements.

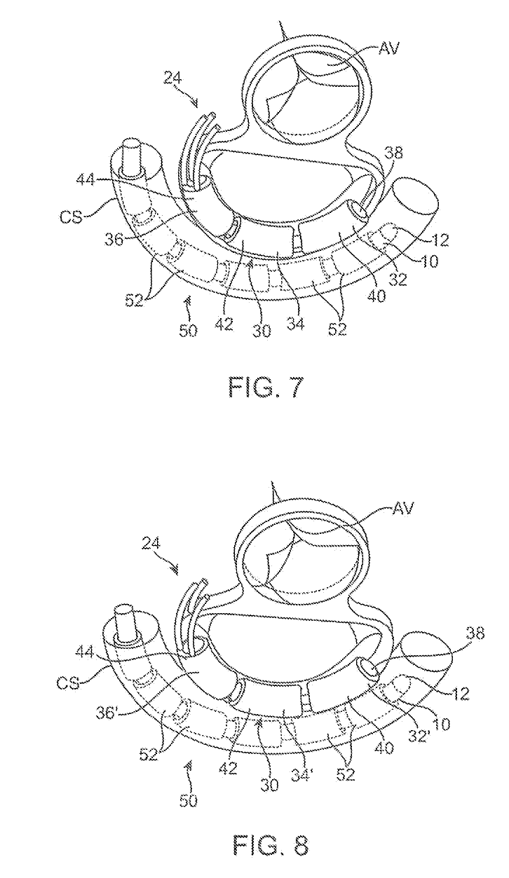

With balloon catheter assembly 30 and magnetic chain catheter 50 each drawn towards one another and securely positioned against the tissue in their respective locations, the expandable member 22 disposed upon the distal tip of guidewire 18 may be deflated and guidewire 18 and/or delivery catheter 14 may be withdrawn proximally through lumen 38 of balloon catheter assembly 30 leaving assembly 30 in position against or along the posterior mitral leaflet PML, as illustrated in FIG. 7. Guiding catheter 10 may also be optionally withdrawn from magnetic chain catheter 50 leaving catheter 50 within the coronary sinus CS.

The one or more balloons 32′, 34′, 36′ may then be desirably inflated or expanded uniformly or in various combinations to buttress the posterior mitral leaflet PML, as described above, and as shown in FIG. 8. Alternatively, balloon catheter assembly 30 may be desirably inflated or expanded prior to the withdrawal of delivery catheter 14 and/or guiding catheter 10. With the inflated or expanded balloons 32′, 34′, 36′ positioned against or upon the inferior surface of the posterior mitral leaflet PML, assembly 30 may function as a support which inhibits or prevents the posterior mitral leaflet PML from prolapsing and further buttresses the posterior mitral leaflet PML such that the proper coaptation of the posterior mitral leaflet PML relative to the anterior mitral leaflet AML is facilitated.

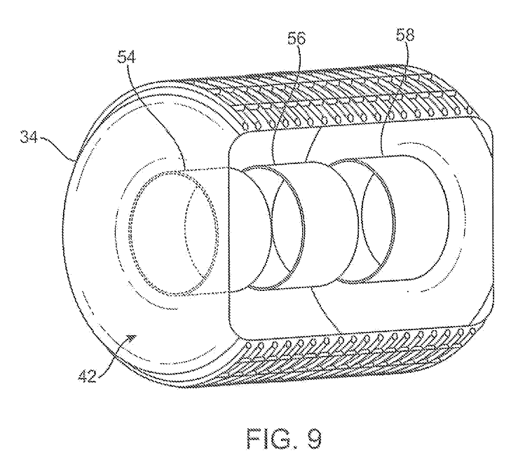

As described above, magnets 40, 42, 44 may be integrated within or along balloon catheter assembly 30. In other variations, multiple magnets may be positioned within a single balloon, as shown in FIG. 9. In this variation, three or more magnets 54, 56, 58 may be aligned in series and configured as circumferentially-shaped or ring magnets which surround the central inflation lumen. These magnets 54, 56, 58 may be positioned within a balloon member 34, as illustrated, or in-between balloon members.

As previously mentioned above, any number of imaging modalities may be utilized, e.g., fluoroscopy, echocardiography, ultrasound, computed tomography (CT), magnetic resonance imaging (MRI), etc. Transesophageal echocardiography in particular may be utilized with any of the variations described herein to provide in vivo imaging during advancement and/or deployment of the devices described herein. Typically, transesophageal echocardiography is performed by utilizing a high-frequency ultrasound transducer mounted on the tip of an endoscope or gastroscope which is passed per-orally into the patient's esophagus and advanced until the ultrasound transducer is adjacent to the patient's heart. Because the posterior portion of the heart is in close proximity to the lower portion of the esophagus, ultrasound images of the interior of the patient's heart may be obtained directly by the transducer.

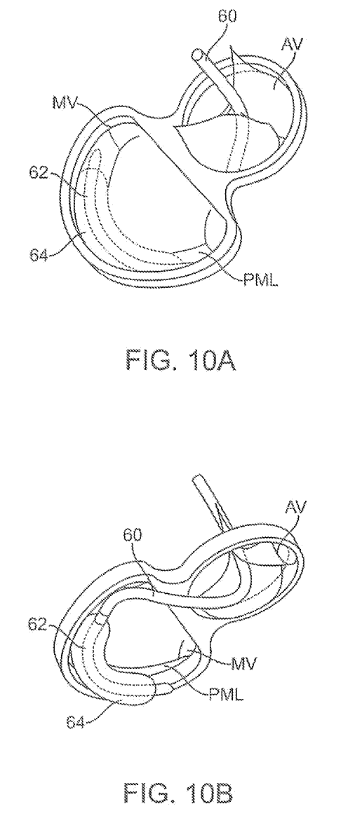

As mentioned above, other alternative inflatable or expandable balloon members may be utilized. FIGS. 10A and 10B illustrate perspective views of a delivery catheter 60 advanced intravascularly, as above, having an inflatable delivery member 62 configured as a single continuous inflatable balloon 64. One or more magnets may also be integrated within the delivery member 62. In such a variation, the single inflatable balloon 64 may be configured to have a length which approximates the posterior mitral leaflet PML such that balloon 64 may be positioned therealong. Once inflated, the balloon 64 may be left in place while secured via magnetic attraction from the magnetic chain catheter 50 (not shown for clarity) positioned within the coronary sinus CS, as above.

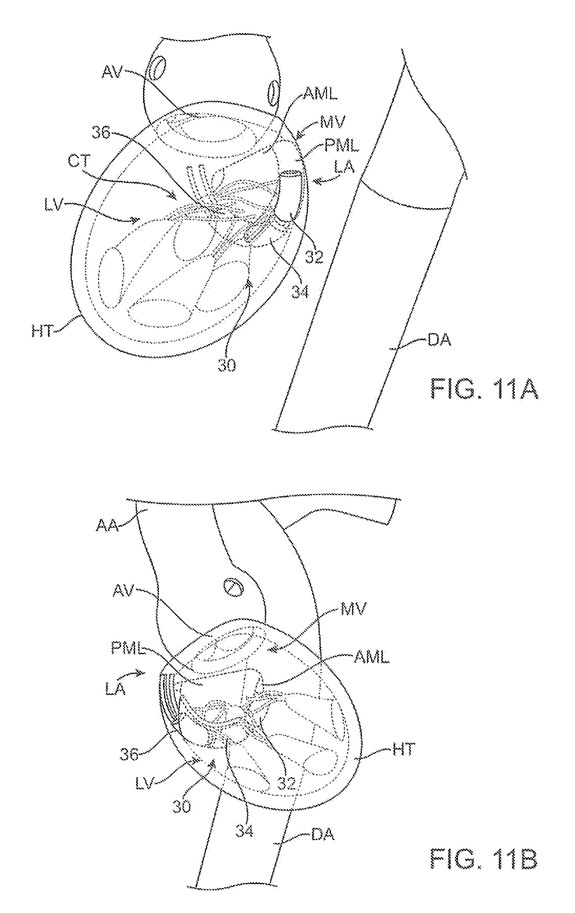

FIGS. 11A and 11B illustrate the inflated balloon catheter assembly 30 positioned along or against the inferior surface of the posterior mitral leaflet PML within the left ventricle LV and inferior to the left atrium LA of the patient heart HT. Also shown are the aortic valve AV, aortic arch AA, and descending aorta DA through which the delivery catheter 14 may be advanced through to access the posterior mitral leaflet PML.

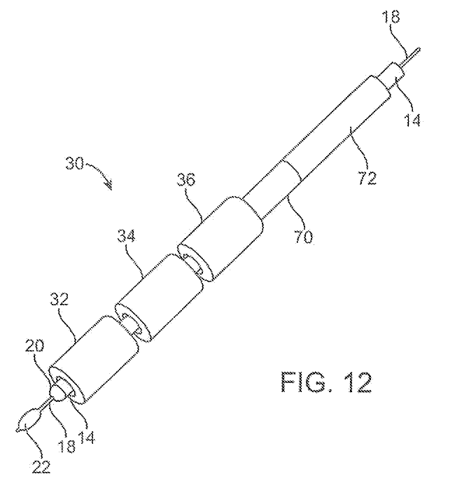

Turning now to the delivery and deployment system in further detail, FIG. 12 illustrates a perspective view of one variation of the system. As shown, the balloon assembly 30 may be attached, coupled, or otherwise removably connected via catheter coupling mechanism 70 to a catheter outer shaft or tubing 72 which may be utilized to advance and deploy balloon assembly 30. Catheter shaft 72 may also define a lumen through which delivery catheter 14 and guidewire 18 may be advanced through during the initial advancement and positioning of delivery catheter 14 relative to the posterior mitral leaflet PML.

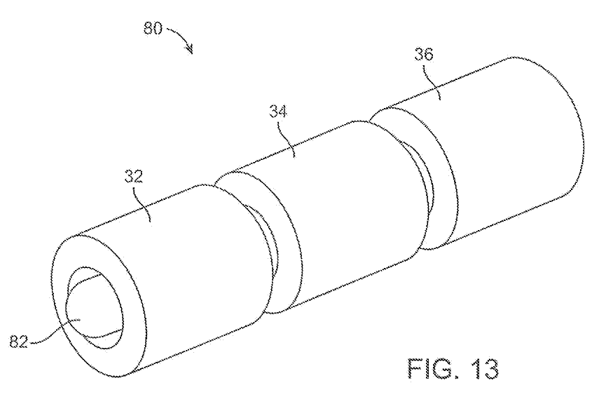

The balloon catheter assembly removably connected to catheter shaft 72 may generally comprise one or more inflatable or expandable balloon members 32, 34, 36 which may be expanded via corresponding inflation lumens routed through catheter shaft 72. However, another variation is shown in the perspective view of FIG. 13 where balloon catheter assembly 80 may utilize a single inflation shaft 82 having an adjustable occluding mechanism to inflate each balloon member independently of one another.

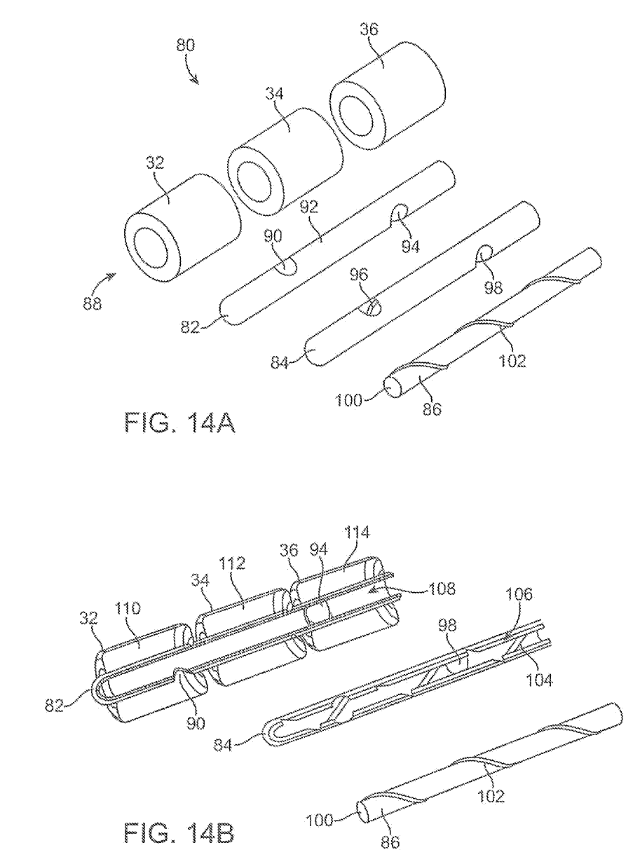

In this particular variation, FIGS. 14A and 14B illustrate exploded assembly and exploded cross-sectional assembly views, respectively, of balloon assembly 80. Each balloon member 32, 34, 36 may define a common lumen 88 therethrough within which a common inflation shaft 82 may be securely positioned. Inflation shaft 82 may define at least a first opening 90 which is in communication within a first balloon interior 110 of first balloon member 32. A second opening 92 defined along inflation shaft 82 may likewise be in communication with a second balloon interior 112 of second balloon member 34 and yet a third opening 94 also defined along inflation shaft 82 may likewise be in communication with a third balloon interior 114 of third balloon member 36. Lumen 108 may be defined through inflation shaft 82 and is common to each of the openings 90, 92, 94.

A separate wall occluding shaft 84 may be slidably positionable within lumen 108 and may further define one or more openings 96, 98 therealong which may be in communication with a common lumen 106 defined through a length of the occluding shaft 84. The interior surface of wall occluding shaft 84 may further define a helical groove or track 104 throughout its length along which an infusion catheter shaft 86 may be advanced therealong. Infusion catheter shaft 86 may accordingly define a helical rail or projection 102 along its outer surface corresponding to the helical track 104 defined along the inner surface of wall occluding shaft 84. Infusion catheter shaft 86 may further define an inflation lumen 100 in communication with a pump and/or externally located fluid or gas reservoir through which the balloon members 32, 34, 36 may be inflated or otherwise expanded.

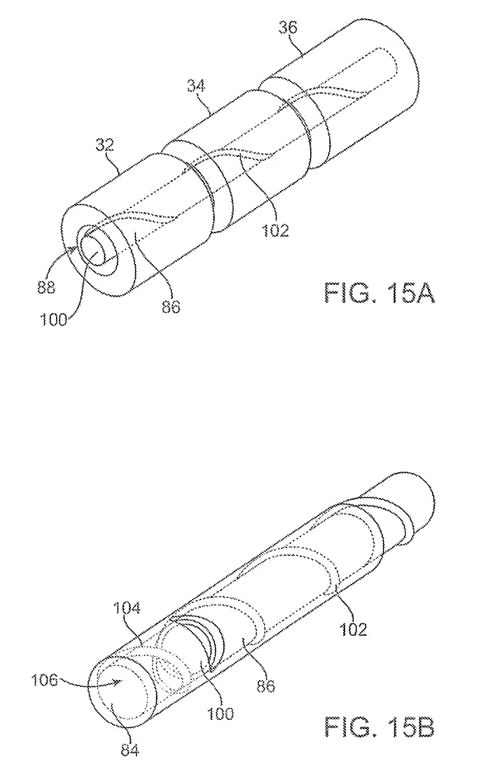

FIG. 15A illustrates a perspective view showing infusion catheter shaft 86 having a helical rail 102 illustratively positioned within lumen 88 of balloon members 32, 34, 36. FIG. 15B illustrates infusion catheter shaft 86 rotatably disposed within wall occluding shaft 84 to illustrate the advancement and retraction mechanism. To advance infusion catheter shaft 86 distally relative to wall occluding shaft 84, infusion catheter shaft 86 may be rotated about its longitudinal axis in a first direction such that helical rail 102 is engaged within the corresponding helical groove 104 and infusion shaft 86 is urged distally within occluding shaft 84. Likewise, to urge infusion shaft 86 proximally relative to occluding shaft 84, infusion shaft 86 may be rotated about its longitudinal axis in the opposite direction such that the engaged helical rail 102 urges the infusion shaft 86 accordingly.

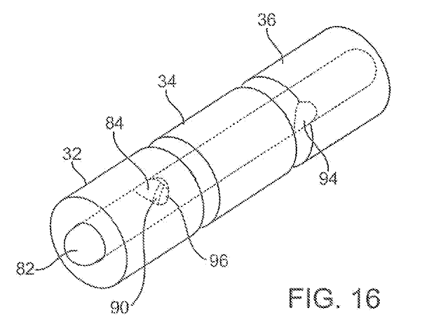

Generally in operation, wall occluding shaft 84 may be translated until its first opening 96 defined along its length is aligned with first opening 90 defined along the length of inflation shaft 82. With the openings aligned, a fluid or gas as described above may be passed through infusion catheter 86 to flow through the aligned openings 90, 96 and into one of the balloon members to inflate or expand the balloon, such as balloon member 32 as shown in FIG. 16. Meanwhile, occluding shaft 84 may also occlude the other openings defined along inflation shaft 82, such as third opening 94 to prevent the infusion of fluids into the remaining balloon members. In this manner, each balloon may be inflated or expanded individually to optionally customize the inflation pattern of the balloon assembly 30.

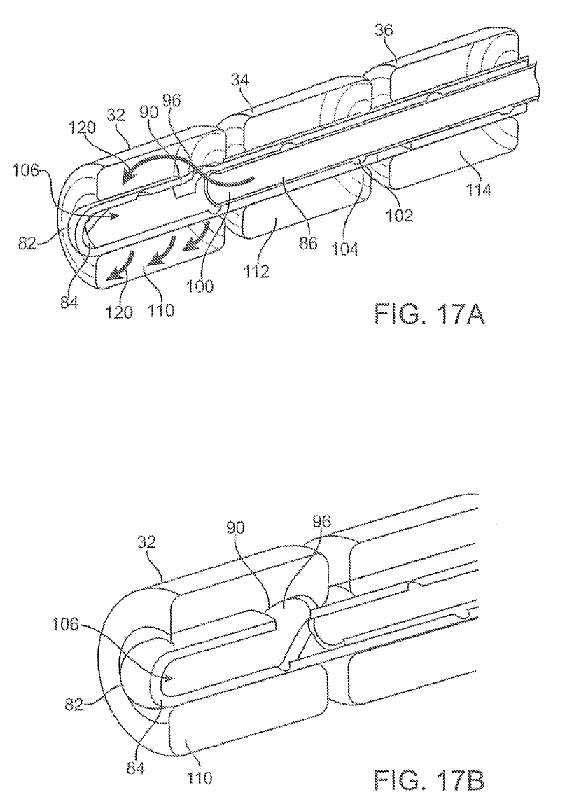

In an example for how each individual balloon member may be optionally inflated or expanded, FIGS. 17A and 17B show partial cross-sectional perspective and detail views, respectively, illustrating one method for inflating first balloon member 32. As described, first opening 96 of wall occluding shaft 84 may be aligned with first opening 90 of inflation shaft 82. With the opening of infusion lumen 100 positioned proximally of the aligned openings 90, 96, fluid or gas 120 may be injected through infusion catheter 86 such that the fluid or gas travels through infusion lumen 100 and directly through the aligned openings 90, 96 and into first balloon interior 110. Wall occluding shaft 84 may be aligned such that the openings leading into second balloon interior 112 or third balloon interior 114 are occluded and prevented from inflating or expanding.

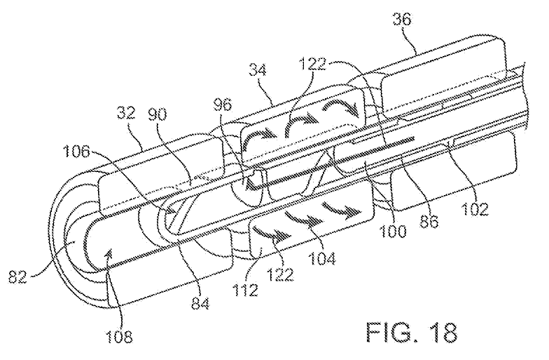

With first balloon member 32 having been inflated, wall occluding shaft 84 may be withdrawn partially to occlude the opening 90 of inflation catheter 82 leading to the first balloon interior 110. Opening 96 of wall occluding shaft 84 may then be aligned with the second opening leading into second balloon interior 112 and infusion catheter 86 may be optionally withdrawn partially by rotating the shaft to engage the helical track. Once aligned and with the first and third openings 90, 94 occluded by shaft 84, the fluid or gas 122 may be passed through infusion lumen 100 such that the second balloon interior 112 is filled accordingly, as shown in FIG. 18.

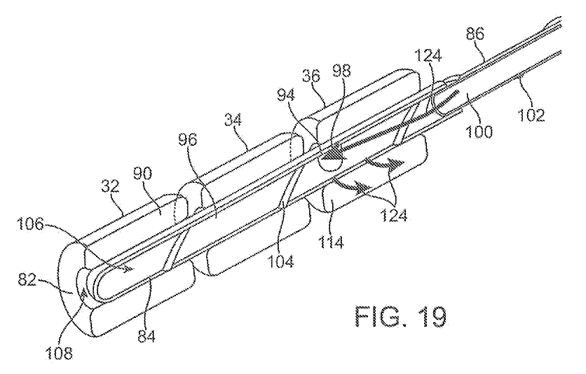

With first and second balloon members 30, 32 filled, the remaining third balloon member 36 may be filled. As illustrated in FIG. 19, wall occluding shaft 84 may be rotated or moved longitudinally such that the opening 98 defined in shaft 84 is aligned with the opening 94 in inflation shaft 82 such that the lumen 106 of infusion catheter 84 is in communication with the third balloon interior 114 while communication with the first and second balloon interiors 110, 112 is precluded. With the openings aligned, infusion catheter 86 may be moved proximally such that the opening of lumen 100 is proximally positioned relative to opening 94 in inflation shaft 82. Once properly aligned, the third balloon interior 114 may be infused with the fluid or gas 124 accordingly.

To facilitate the selective occlusion and opening of each of the openings leading to the balloon members 32, 34, 36 with respect to wall occluding shaft 84, each of the openings 90, 92, 94 located along inflation catheter 82 may be positioned at varying angles relative to one another. Accordingly, each opening 90, 92, 94 may be off-set with respect to one another in such a manner that if wall occluding shaft 84 were rotated about its longitudinal axis, each opening would become un-occluded by shaft 84 one at a time while the remaining two openings remain occluded at any given point. In this manner, any one of the balloon members may be inflated or expanded independently from one another.

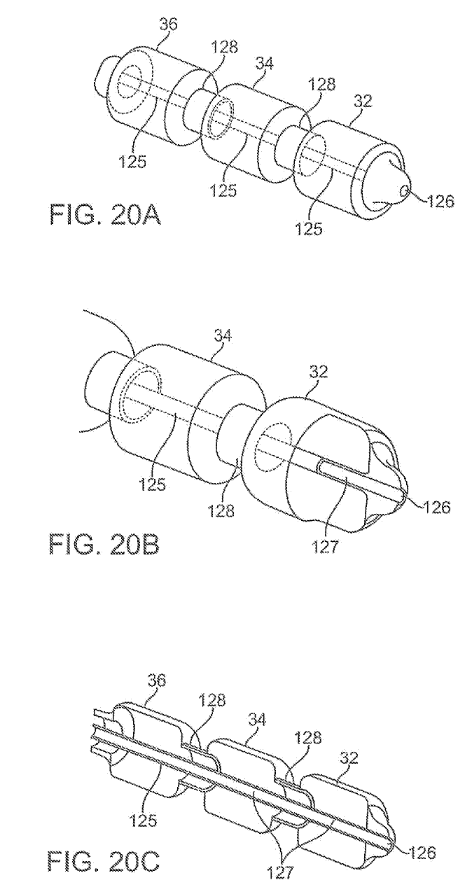

Another variation of the balloon assembly is illustrated in the perspective views of FIGS. 20A and 20B which show balloon members 32, 34, 36 having tubular channel 125 passing through each of the members and terminating within first balloon member 32. Tubular channel 125 may define a guidewire lumen 127 passing through tubular channel 125 and terminating at distal opening 126 such that a guidewire may be passed through the entire length of the balloon assembly to facilitate placement and/or guidance of the device to the desired location. FIG. 20C further illustrates a cross-sectional perspective view of balloon members 32, 34, 36 and guidewire lumen 127 passing through the length of the assembly. Each balloon member may be further interconnected to one another via connecting collars 128 to provide a structural connection between the members as well as to provide sealing for inflation of the members. Collars 128 may also contain the magnetic material for attraction to the complementary magnetic chain, as described above.

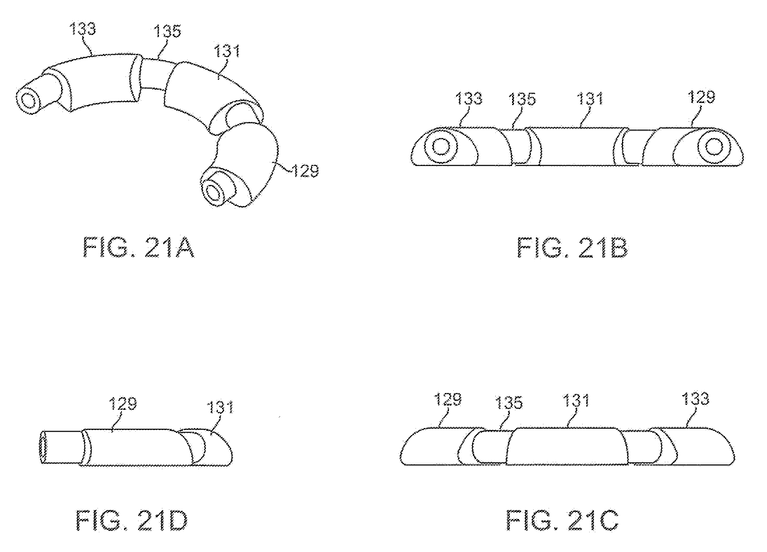

FIG. 21A shows a perspective view of yet another variation of the balloon assembly where respective first, second, third balloon members 129, 131, 133 may be interconnected via inflation shaft 135, as above, and where the balloon members 129, 131, 133 are not symmetrically shaped. FIGS. 21B and 21C illustrate front and rear views and FIG. 21D illustrates an end view of balloon members 129, 131, 133 which are configured to each have a flattened surface. The flattened surface allows for the inflated balloon members to occupy less space when positioned inferior to the mitral leaflet while the curved or arcuate portion of the balloon members may be placed into contact against the leaflet to provide a contoured contact surface.

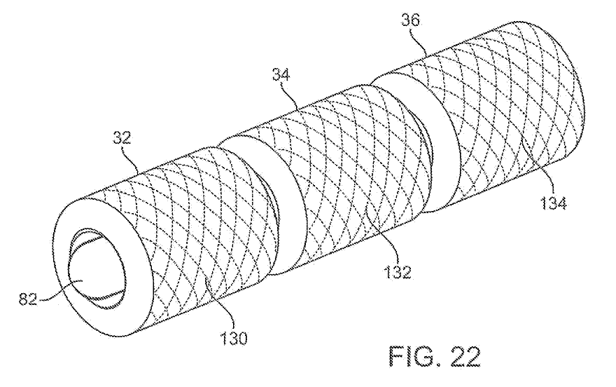

Aside from a distensible or expandable balloon membrane which is inflated or expanded by an infusion of fluid or gas, alternative variations for the balloon membrane may be utilized which remain in an enlarged configuration once expanded. For instance, FIG. 22 illustrates another variation of the balloon assembly where one or more of the balloon membranes may include an expandable scaffold 130, 132, 134. Such a scaffold may be integrated within or upon the balloon membrane as an expandable woven or braided structure. Moreover, the scaffold 130, 132, 134 may be formed of a polymeric or metallic material such as stainless steel or from a self-expandable or shape memory material such as Nickel-Titanium alloy, where the scaffold 130, 132, 134 may self-expand when released from the constraints of a catheter. Additionally, the scaffold 130, 132, 134 may be reconfigured into its expanded configuration upon the infusion of a fluid or gas into the respective balloon members. Once expanded, the scaffold 130, 132, 134 may be configured to retain its shape regardless if the fluid or gas were to leak from the balloon interiors.

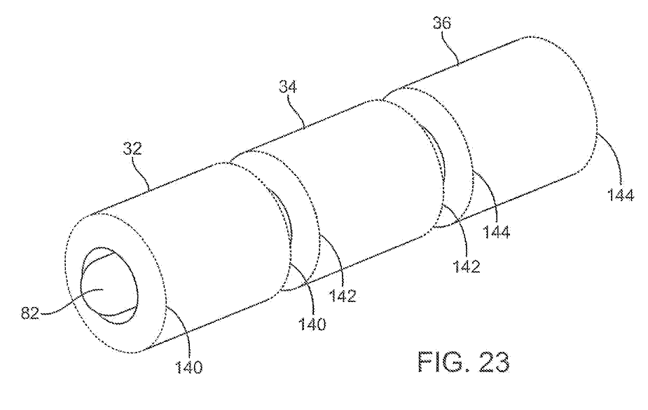

In yet another variation of the balloon assembly, balloon members 32, 34, 36 may have one or more expandable rings 140, 142, 144, respectively, integrated within the balloons. For instance, in the variation illustrated in FIG. 23, the one or more expandable rings may be positioned along one or both ends of each balloon member to provide integrity to the expanded configuration. Accordingly, multiple expandable rings may be integrated along the balloon length as desired and each expandable ring may be comprised of any of the polymeric, metallic, or alloy materials, as described above.

In yet another variation, FIGS. 24A and 24B illustrate perspective and partial cross-sectional perspective views, respectively, of a balloon variation 135 having an integrated stent-like structure 136. The expandable stent 136 may comprise any number of expandable stent structures expandable from a low-profile delivery configuration to an expanded deployment configuration. Stent 136 may be integrated between a distensible outer cover 138 and an inner liner 139 such that when fluids or gas are infused, e.g., through one or more infusion ports 142 defined along an infusion lumen 141, the stent 136 may be expanded into its deployed configuration. Moreover, stent 136 may be comprised of any of the materials described above, especially shape memory alloys such that when expanded, stent 136 may retain its expanded structure against the tissue surface.

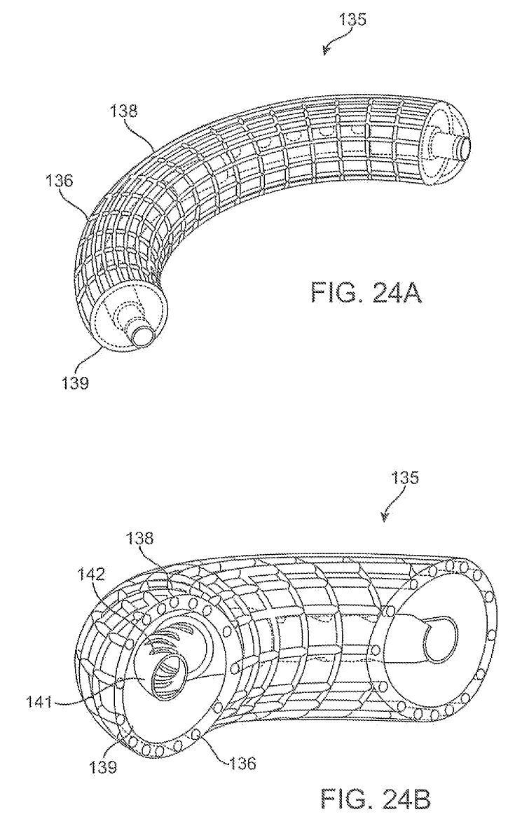

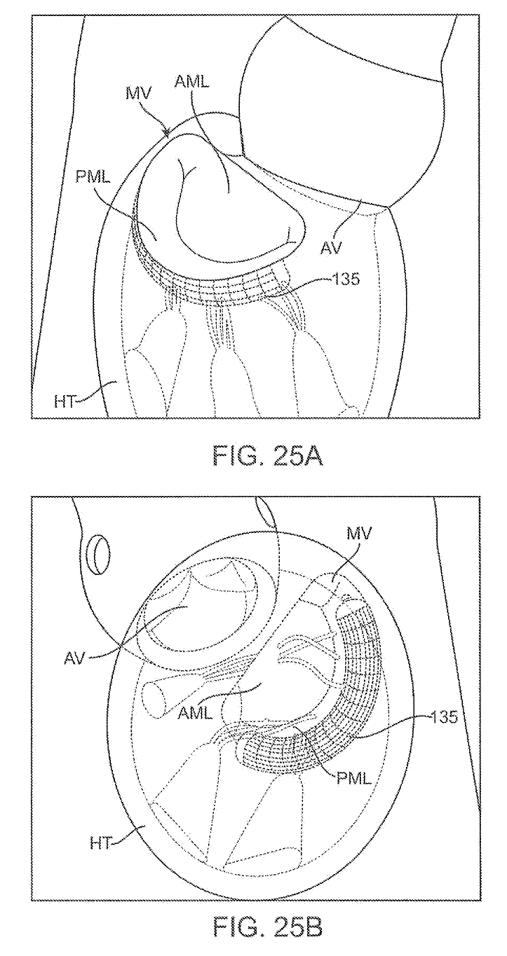

FIGS. 25A and 25B illustrate perspective superior and inferior views, respectively, of the balloon variation 135 deployed against or along the inferior surface of the posterior mitral leaflet PML. Although illustrated as a singular continuous expandable balloon structure, balloon 135 may alternatively be configured into multiple balloon members each having an expandable stent structure, if so desired.

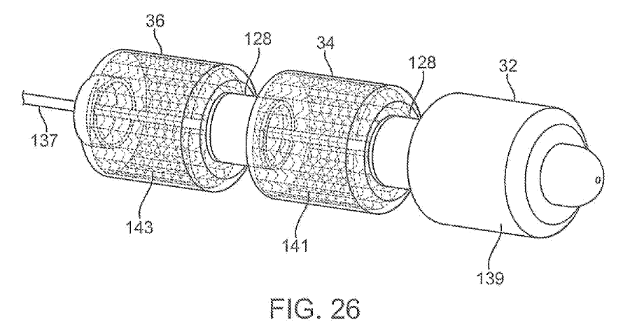

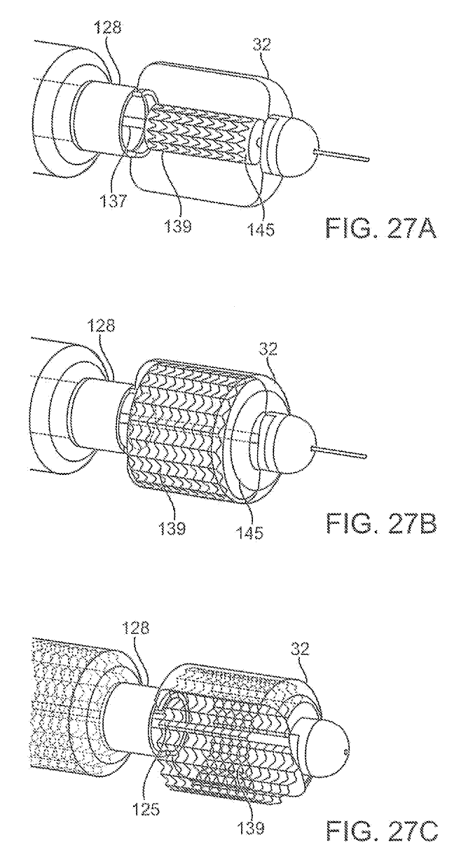

FIG. 26 illustrates a perspective view of such a variation where each balloon member 32, 34, 36 has its own respective expandable stent structure 139, 141, 143 positionable within each member to provide structural support for the balloon member in its expanded configuration. One or more the stent structures may be deployed via deployment catheter 137 which may have one or more respective stent deployment balloons and which may passed along a catheter or member, such as tubular channel 125, through each balloon member 32, 34, 36. A partial cross-sectional view of first balloon member 32 is shown in FIG. 27A which illustrates deployment catheter 137 having first deployable stent 139 disposed over first stent delivery balloon 145 and contained in its unexpanded configuration entirely within first member 32. Upon inflation of first stent delivery balloon 145, first stent 139 may be expanded, as shown in FIG. 27B, to either expand first member 32 into its expanded shape or to maintain an already inflated first member 32. In either case, once first stent 139 has been expanded, first stent delivery balloon 145 may be deflated and removed from first balloon member 32 to maintain the inflated or expanded configuration of first member 32, as shown in FIG. 27C.

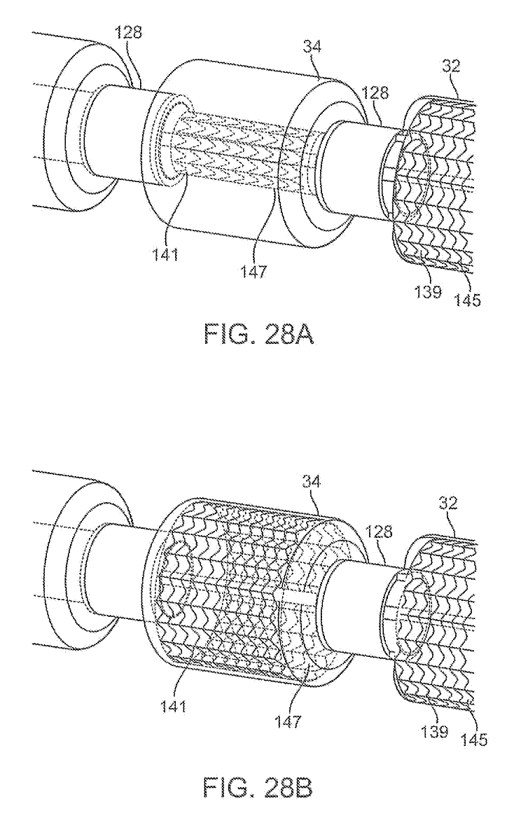

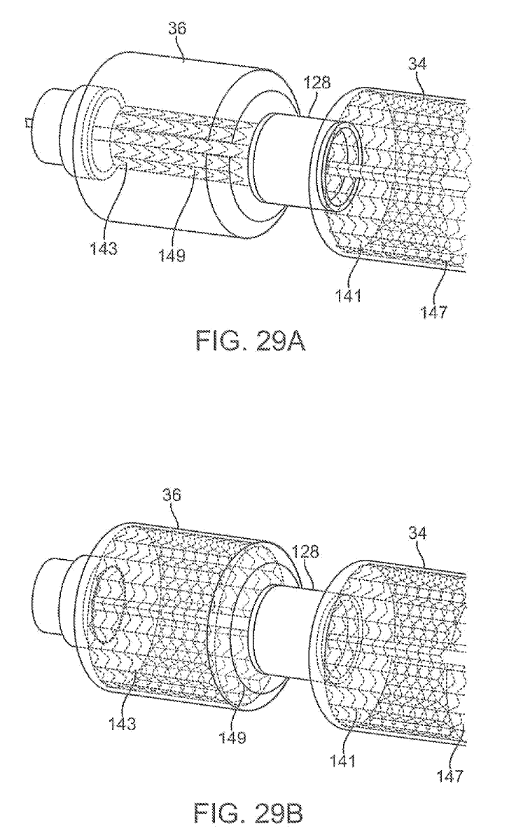

FIGS. 28A and 28B show perspective views of second deployable stent 141 positioned along second stent delivery balloon 147 within second balloon member 34. Upon inflation of second stent balloon 147, second stent 141 may be expanded to provide structural support of second balloon member 34, as shown in FIG. 28B. Likewise, third deployable stent 143 positioned on its respective third stent delivery balloon 149 within third balloon member 36 may be inflated to maintain the inflated or expanded configuration of third member 36, as illustrated in FIGS. 29A and 29B. Although illustrated as having each balloon member 32, 34, 36 inflated prior to stent expansion, balloon inflation and stent expansion may be accomplished simultaneously or stent expansion may cause the reconfiguration of the respective balloon members 32, 34, 36. Moreover, each stent delivery balloon 145, 147, 149 may be inflated sequentially or simultaneously to expand or support each respective balloon member 32, 34, 36 in a sequential or simultaneous manner, depending upon the desired results.

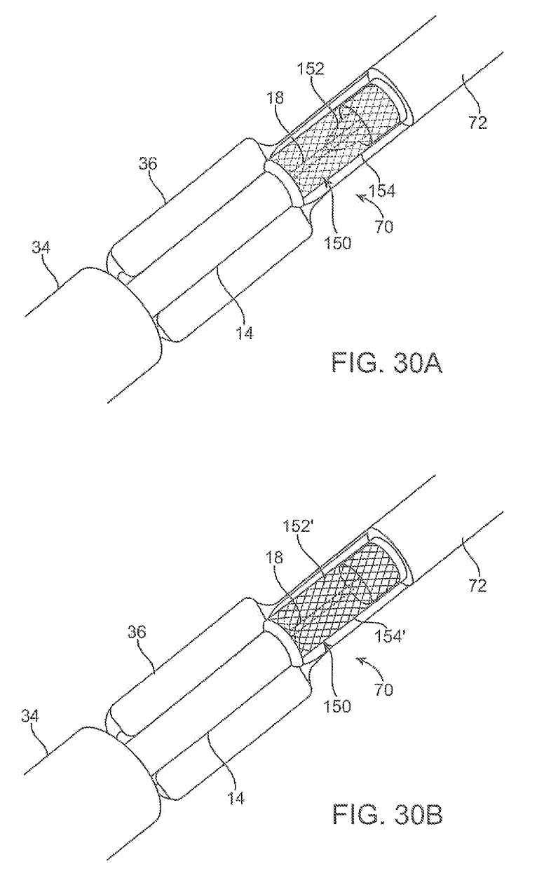

Turning now to deployment mechanisms of the balloon assemblies, FIG. 30A illustrates one variation of a detachment mechanism. As shown, balloon assembly 30 may be coupled to catheter outer shaft 72 via a coupling lumen 150 positioned at a proximal end of assembly 30. Catheter outer shaft 72 may have guidewire 18 and delivery catheter 14 extending from outer shaft 72 within assembly 30. Positioned proximally of delivery catheter 14 along guidewire 18 and housed within coupling lumen 150 is release mechanism 152, which in this particular variation is an expandable balloon. Also housed within coupling lumen 150 and attached to assembly 30 is a coupling mechanism 154, which in this variation is an expandable stent structure or crimp, which physically couples assembly 30 to catheter outer shaft 72.

During intravascular delivery, the balloon assembly 30 may remain securely attached to catheter outer shaft 72. However, during deployment and release of the balloon assembly 30 within the left ventricle, release mechanism balloon 152 may be inflated or expanded 152′ such that it radially contacts coupling mechanism stent 154′ and urges it into an outward radial direction to release the coupling stent 154′ from catheter outer shaft 72 and to thereby release balloon assembly from catheter shaft 72, as shown in FIG. 30B.

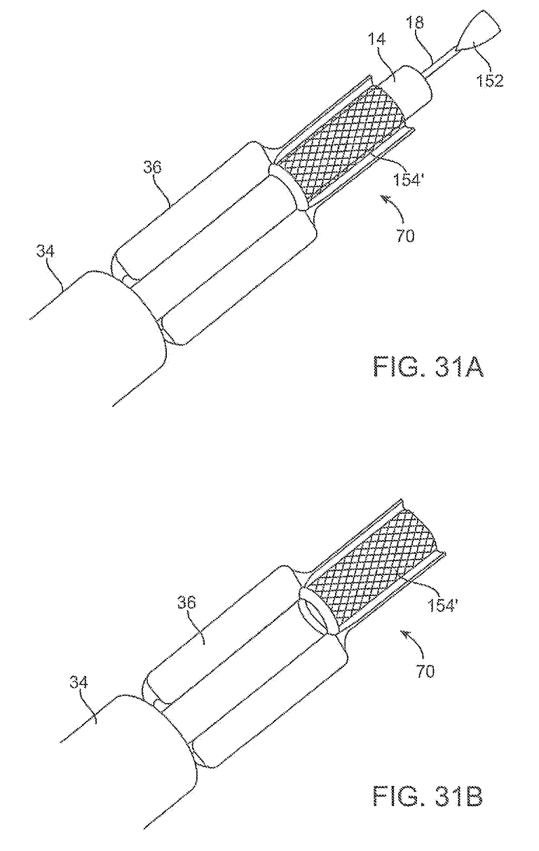

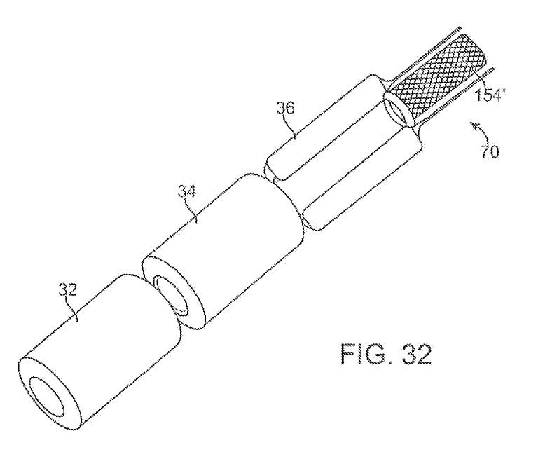

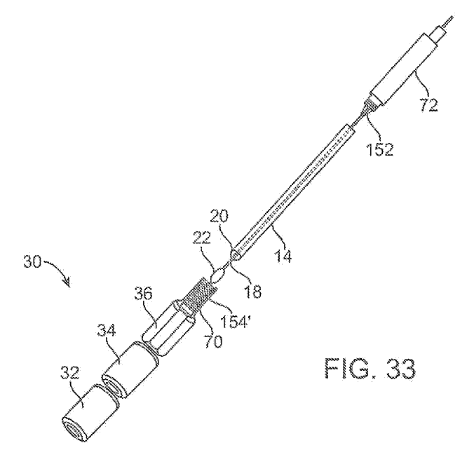

With coupling mechanism stent 154′ disengaged, release mechanism balloon 152 may be deflated to allow catheter shaft 72 and delivery catheter 14 to be withdrawn from balloon assembly 30, as shown in FIG. 31A. With the complete withdrawal of the catheter shaft 72, balloon assembly 30 may be left in place against or along the posterior mitral leaflet PML, as illustrated in FIG. 31B. FIG. 32 illustrates a partial cross-sectional view of the balloon assembly having delivery catheter 14 removed and FIG. 33 illustrates a perspective assembly view of the coupling mechanism released with the delivery catheter 14 and catheter shaft 72 being removed from the balloon assembly 30.

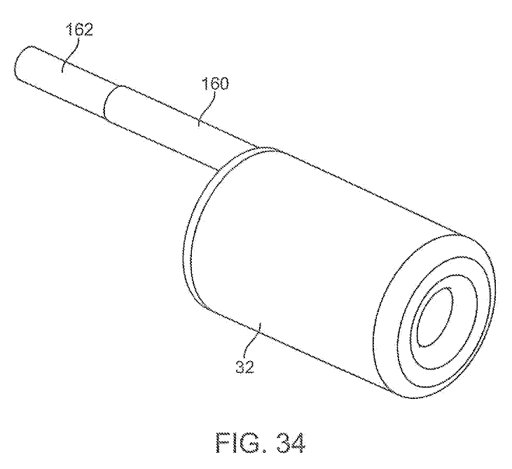

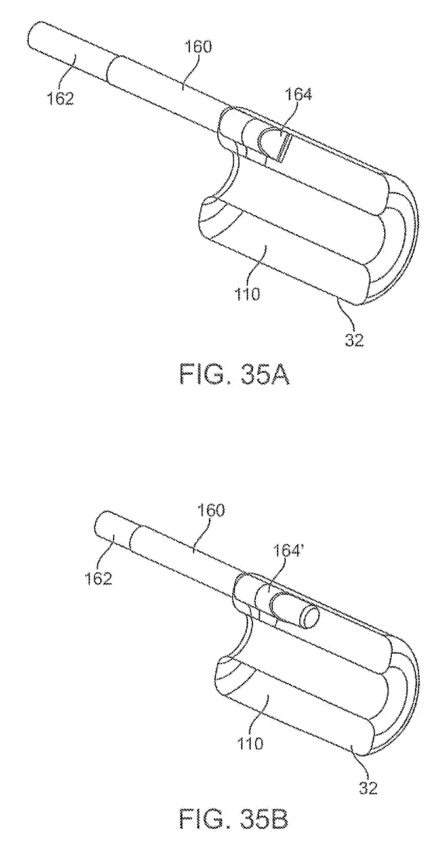

In yet another variation for inflating or infusing a balloon member, FIG. 34 shows a perspective view of a single balloon member 32 for clarity with an infusion lumen 160 in communication with the balloon member 32. An infusion catheter 162 which is translatable relative to infusion lumen 160 may be positioned therewithin. FIG. 35A illustrates a partial cross-sectional view showing the balloon 32 with infusion lumen 160 having a unidirectional valve 164 attached to a distal end of the lumen 160 within balloon member 32. To inflate balloon member 32, infusion catheter 162 may be translated distally through lumen 160 until the distal end of catheter 162 breaches valve 164, thereby forcing the valve into an opened configuration 164′.

With the distal tip of catheter 162 positioned within balloon interior 110, a fluid or gas, as above, may be infused into the balloon member 32 to inflate it, as shown in FIG. 35B. Once the balloon member 32 has been desirably inflated, catheter 162 may be withdrawn allowing valve 164 to close and prevent leakage of the infused fluid or gas. Aside from the infusion of fluids or gases, practically any biocompatible polymer, co-polymer, and/or their blends, such as swellable hydrogels, which are formable or settable may be utilized, if so desired.

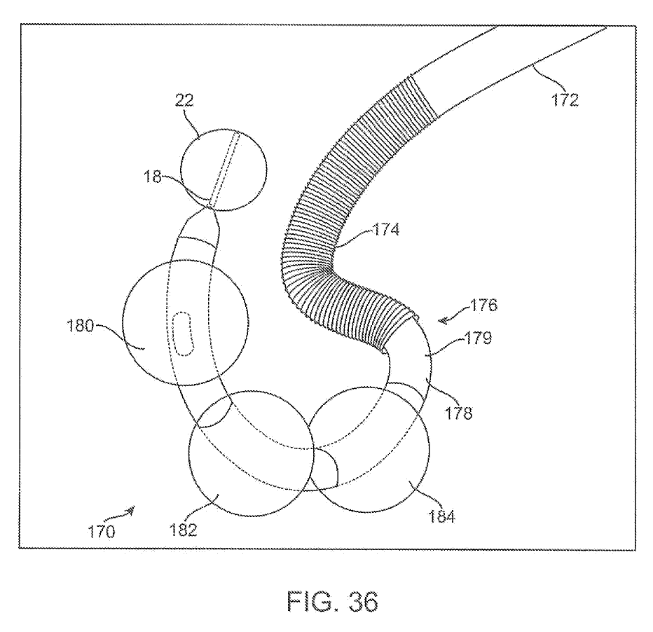

In yet another variation of a balloon member assembly, FIG. 36 shows a perspective detail view of inflation assembly 170 coupled to a delivery catheter 179 via release mechanism 178, as described above. In this variation, first, second, and third respective inflatable balloon members 180, 182, 184 may be spherically shaped and individually inflated via a separate inflation lumen, as mentioned previously and as further described below. Inflation assembly 170 may further define a guidewire lumen through the assembly through which guidewire 18 and inflatable or expandable member 22 may be delivered through to assist in initially anchor and placing the assembly. Delivery sheath 172 may define sheath lumen 176 through which inflation assembly 170 may be advanced may also comprises an articulatable section 174 near or at its distal end. Articulatable section 174 may comprise a portion integrated with a shape memory alloy, e.g., nickel-titanium alloy (Nitinol), such that when sheath 172 is positioned intravascularly within the heart of a patient, section 174 may self-configure or bend (either via shape memory or via one or more pullwires) to guide the guidewire 18 and/or inflation assembly 170 to the target location.

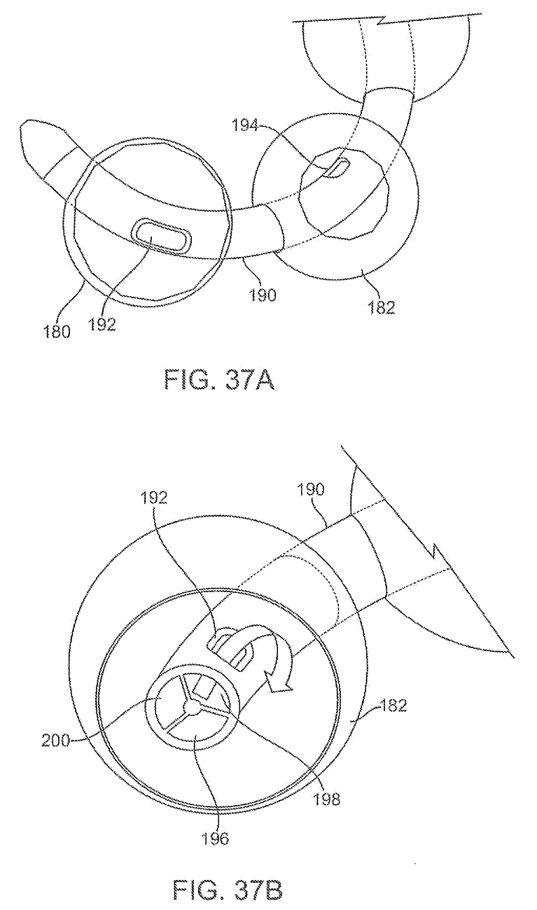

FIG. 37A shows a partial cross-sectional perspective view of first and second balloon members 180, 182 positioned along inflation shaft 190 and illustrating the inflation ports within each member for inflating with a fluid, as described above. For example, first balloon member 180 may be in fluid communication with a fluid reservoir through first inflation port 192 while second balloon member 182 may be in fluid communication through second inflation port 194. Third balloon member 184 is similarly in communication through an inflation port. Rather than utilizing a common inflation lumen for each of the balloon members with a control mechanism, each balloon member may instead have its own respective inflation lumen to independently control the inflation and/or deflation of each balloon member.

FIG. 37B shows a perspective view of a cross-section of inflation shaft 190 illustrating the respective first, second, and third inflation lumens 196, 198, 200 defined therethrough. Inflation fluid may be pumped through one or more the lumens 196, 198, 200 depending upon which balloon member is to be inflated. Moreover, although three separate inflation lumens are shown, lumens numbering two or greater than three may alternatively be defined through inflation shaft 190, depending upon the number of balloon members to be inflated or utilized in the assembly.

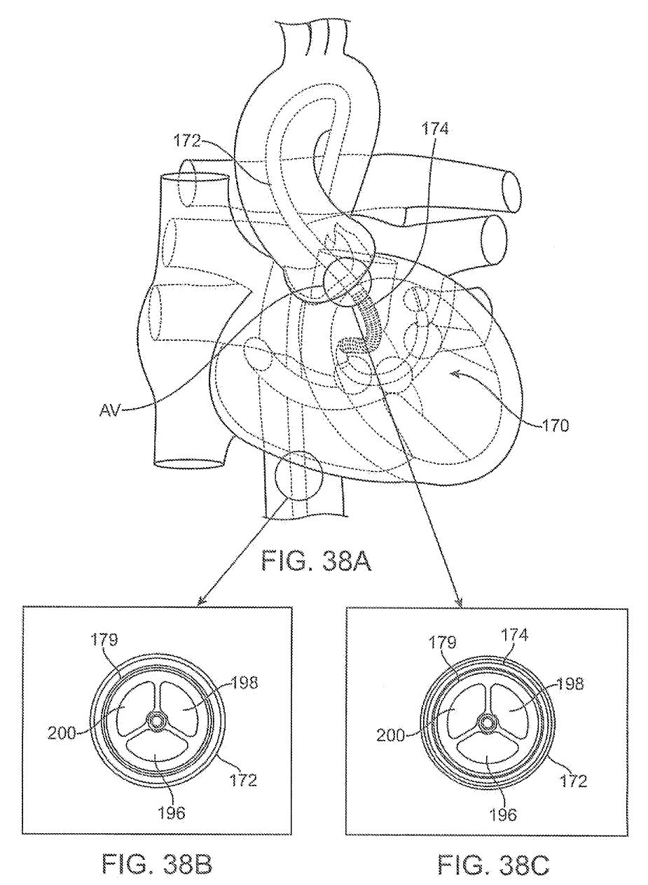

A perspective view of one possible approach for advancing the sheath 172 through the aortic arch and aortic valve AV and within the left ventricular chamber is shown to illustrate a configuration of the delivery catheter 179 having the multiple inflation lumens in communication with the inflation assembly 170, as shown in FIG. 38A. As illustrated in the cross-sectional view of the system along a portion of the catheter proximal to the inflation assembly 170, FIG. 38B shows delivery catheter 179 contained within sheath 172 and the separated inflation lumens 196, 198, 200. FIG. 38C illustrates a cross-sectional view of the system proximal to the inflation assembly 170 showing the delivery catheter 179 contained within sheath 172 and articulatable section 174 also positioned at least partially within sheath 172 and deployable therefrom.

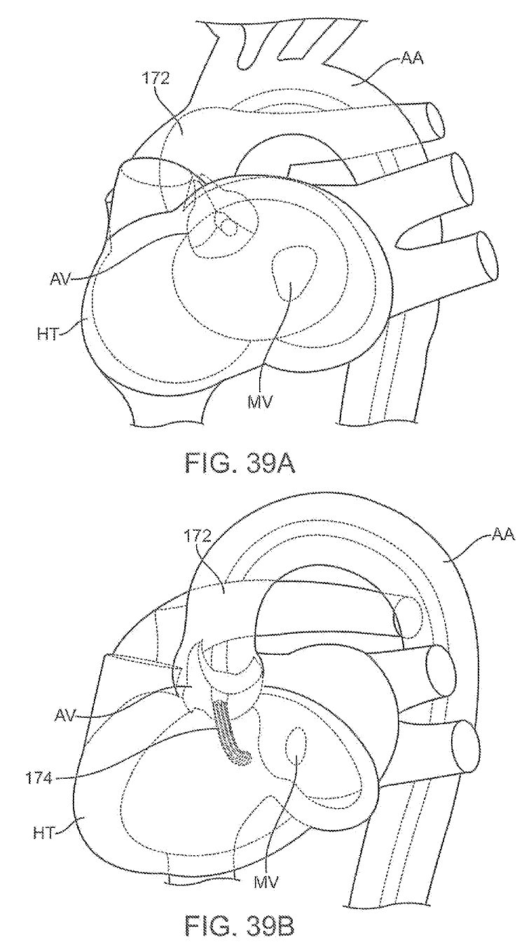

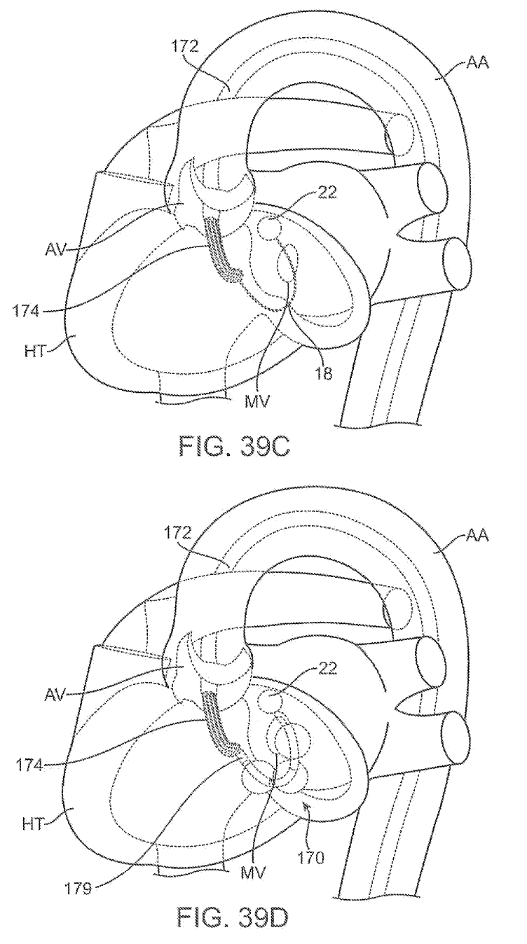

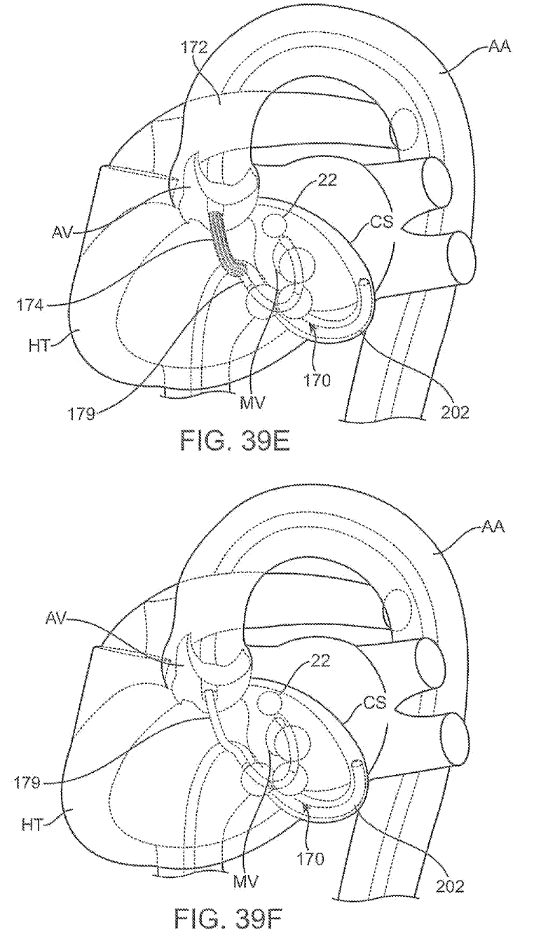

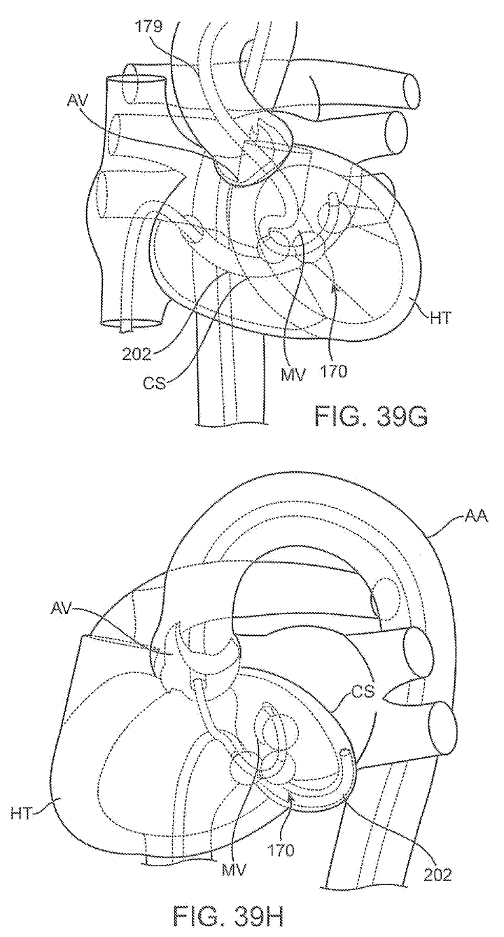

FIGS. 39A to 39H illustrate perspective views of another method for delivering and positioning an inflation assembly inferiorly to a posterior mitral leaflet of the mitral valve MV within a patient heart HT. As shown in FIG. 39A, sheath 172 may be advanced intravascularly through the patient body and into the left ventricular chamber via the aortic arch AA and through the aortic valve AV. The sheath 172 may be guided via a guidewire advanced prior to insertion of sheath 172. With sheath 172 positioned through the aortic valve AV, the delivery catheter 179 may be advanced through sheath 172 until the articulatable section 174 is advanced beyond the distal end of sheath 172 and into the ventricular chamber, as shown in FIG. 39B. As articulatable section 174 becomes unconstrained from sheath 172, it may bend or curve into a predetermined configuration such that its distal end is directed towards the mitral valve MV or the tissue region surrounding the mitral valve MV.

With articulatable section 174 desirably positioned, guidewire 18 may be advanced through the delivery catheter such that guidewire 18 is directed along inferiorly within the ventricular chamber around the posterior portion of the mitral valve MV. Once guidewire 18 has been directed sufficiently around the valve, balloon 22 may be inflated to temporarily anchor a position of the guidewire 18 and delivery catheter relative to the mitral valve MV, as shown in FIG. 39C. Inflation assembly 170 may then be advanced out of sheath 172 along guidewire 18 with the balloon members in their deflated or unexpanded state. With inflation assembly 170 desirably positioned along guidewire 18 relative to the mitral valve MV posterior leaflet, one or more of the balloon members may be inflated or expanded utilizing any of the mechanisms described herein until the posterior leaflet is sufficiently supported, as illustrated in FIG. 39D.

A second catheter, e.g., guiding catheter 202 having a magnet chain as described above, may be introduced and intravascularly advanced along another vascular route, e.g., via the inferior vena cava or superior vena cava, into the right atrial chamber, and into the coronary sinus CS. The magnet chain and guiding catheter 202 may be advanced along the coronary sinus CS until the magnet chain is positioned proximate or adjacent to the mitral valve MV, as described above and as illustrated in FIG. 39E. The magnetic attraction between the guiding catheter 202 and inflation assembly 170 may accordingly draw the two towards one another to anchor the inflation assembly relative to the mitral valve MV. With inflation assembly 170 anchored, outer sheath 172 may be removed, as shown in FIG. 39F, and delivery catheter 179 may be detached from inflation assembly utilizing any of the mechanisms described herein and removed from the patient body leaving inflation assembly 170 against the mitral valve MV. FIGS. 39G and 39H show alternative views of the inflation assembly 170 and guiding catheter 202 relative to the mitral valve MV.

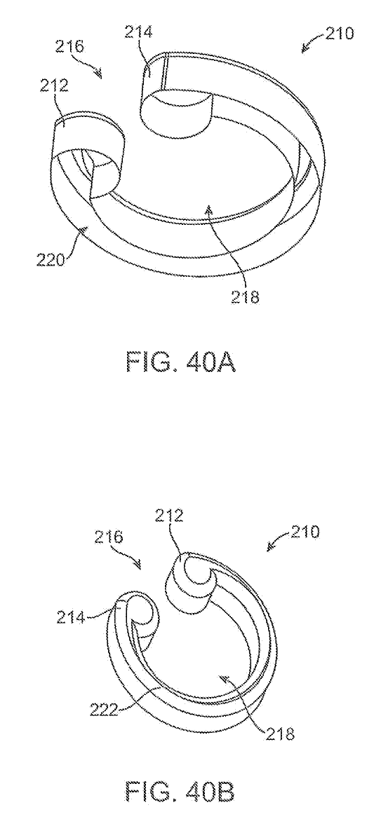

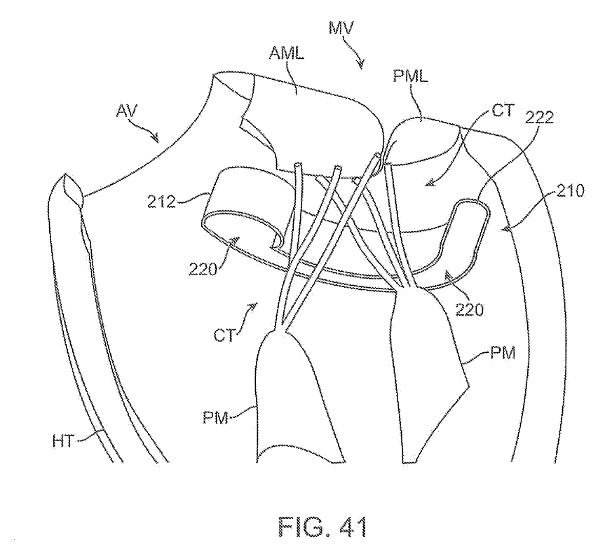

Yet another example of an alternative apparatus which may be utilized to support the mitral valve, particularly the posterior mitral leaflet, in a frozen or immobile position to facilitate the proper coaptation of the posterior and anterior mitral leaflets is illustrated in the perspective view of FIG. 40A. As shown, a device configured as a split ring 210 may have two terminal atraumatic ends 212, 214 in apposition to one other separated by a split 216. The ring 210 may have a central opening 218 defined by the partial circumferential shape of the ring 210. Ring 210 may further form an open channel 220 which is defined around the length of the ring 210. Channel 220 may be enclosed along a top side of the ring 210 by a presentation surface 222, as shown in the alternate perspective view of FIG. 40B.

In one variation, ring 210 may be configured in a circular ring shape, while in other variations, ring 210 may be configured in an elliptical or oval shape. In yet other variations, ring 210 may be configured in a shape which conforms to or tracks at least the shape of the posterior mitral leaflet PML. Moreover, ring 210 may be comprised of a variety of materials; for instance, ring 210 may be fabricated from metallic or polymeric materials. Examples can include shape memory materials, such as a Nickel-Titanium alloy like Nitinol, or various shape memory polymers. Additionally, ring 210 may be coated with a polymeric material or infused with a drug or agent which inhibits the formation of thrombus.

In placing ring 210, the apparatus may be situated within the left ventricle and inferior to the mitral valve MV such that ring 210 is placed at least partially around the chordae tendineae CT supporting the mitral leaflets, as illustrated in the partial cross-sectional view of FIG. 41. In such a placement, the chordae tendineae CT pass through central opening 218 and atraumatic ends 212, 214 may at least partially enclose the chordae tendineae CT therebetween Presentation surface 222 may be orientated to lie directly adjacent to and along the posterior mitral leaflet PML such that channel 220 is oriented to face away from the mitral valve MV and towards the papillary muscles PM.

Ring 210 may also be delivered in a variety of methods. In one variation, ring 210 may be implanted within the left ventricle via an open surgical procedure. Alternatively, ring 210 may be delivered percutaneously via an intravascular approach, in which case ring 210 may be configured into an elongated and compressed low-profile configuration within a delivery-catheter. In this variation, ring 210 may be fabricated from a shape memory alloy or polymeric material. As the catheter is advanced into the left ventricle utilizing any of the approaches described above, a pusher mechanism may urge the ring 210 around the chordae tendineae CT such that as ring 210 is freed from the constraints of the delivery catheter, ring 210 may reconfigure itself into its ring shape around the chordae tendineae CT.

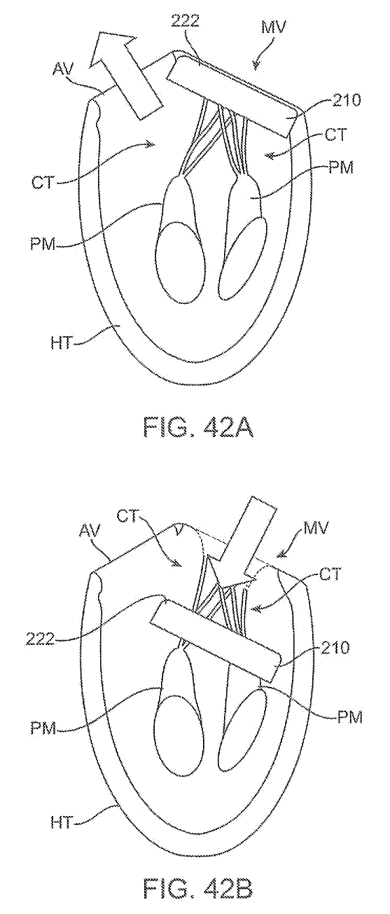

In use, because the chordae tendineae CT may loosely pass through central opening 218, ring 210 may freely slide in vivo along the chordae tendineae CT while retained by the atraumatic ends 212, 214. During systole, as illustrated in FIG. 42A, the left ventricle, contracts urging blood through the aortic valve AV, as indicated by the arrow. Because of the tissue contraction and forced blood flow, which may be captured in part by channel 220, ring 210 may be urged to slide along the chordae tendineae CT into a superior position where presentation surface 222 is urged or pressed against the posterior mitral leaflet PML of the mitral valve MV. As presentation surface 222 is pressed against the mitral valve MV, the posterior mitral leaflet PML may be supported by the ring 210 in inhibiting or preventing prolapse of the leaflet.

During diastole as the ventricle relaxes, ring 210 may freely slide along the chordae tendineae CT into an inferior position towards the papillary muscles PM, as illustrated in FIG. 42B. As the mitral valve MV leaflets are no longer supported by ring 210, blood may freely flow through the mitral valve MV to fill the left ventricle chamber. As the heart HT undergoes systole, ring 210 may slide back towards the mitral valve MV where the process may be repeated.

The applications of the devices and methods discussed above are not limited to the treatment of mitral valves but may include any number of further treatment applications. Other treatment sites may include other areas or regions of the body such as various pulmonary valves, arterial valves, venous valves, tricuspid valves, etc. Alternative combinations between features of the various examples and illustrations, as practicable, as well as modification of the above-described assemblies and methods for carrying out the invention, and variations of aspects of the invention that are obvious to those of skill in the art are intended to be within the scope of the claims.

What is claimed is:

1. A valve leaflet support system, comprising:
at least one support member sized for intravascular delivery and having a low-profile delivery configuration and an expanded deployed configuration,
wherein the at least one support member is further sized for placement against or along an inferior surface of a posterior mitral leaflet.

2. The system of claim 1 further comprising at least one attractive element having a first magnetic polarity coupled to the at least one support member.

3. The system of claim 2 further comprising an anchoring element with at least one attractive element having a second magnetic polarity opposite to the first magnetic polarity and sized for placement in a coronary sinus of a patient.

4. The system of claim 3 wherein the anchoring element comprises a magnetic chain catheter having a plurality of magnets each with the second magnetic polarity.

5. The system of claim 3 further comprising a guiding catheter over which the anchoring element is translatable.

6. The system of claim 5 further comprising a magnetic element positioned on a distal tip of the guiding catheter.

7. The system of claim 1 further comprising a guidewire along which the at least one support member is advanceable.

8. The system of claim 1 further comprising a delivery catheter along which the at least one support member is advanceable.

9. The system of claim 8 further comprising a magnetic element positioned on a distal tip of the delivery catheter.

10. The system of claim 8 further comprising a guidewire along which the delivery catheter is advanceable.

11. The system of claim 10 further comprising an inflatable element disposed on a distal tip of the guidewire.

12. The system of claim 1 wherein the at least one support member is comprised of a single expandable balloon having a length sized for placement against or along the inferior surface of the posterior mitral leaflet.

13. The system of claim 12 further comprising an expandable scaffold or stent integrated within or upon a membrane of the expandable balloon.

14. The system of claim 1 wherein the at least one support member comprises at least three expandable balloons aligned in series.

15. The system of claim 14 further comprising an inflation shaft interconnecting the at least three expandable balloons, the inflation shaft defining a corresponding inflation opening therealong in communication with each expandable balloon.

16. The system of claim 15 further comprising a wall occluding shaft which is slidably movable within the inflation shaft, the wall occluding shaft defining at least one opening therealong for alignment with at least one inflation opening.

17. The system of claim 16 further comprising an infusion catheter rotatably movable via a helical track within the wall occluding shaft, the infusion catheter in fluid communication with a fluid reservoir.

18. The system of claim 15 wherein the inflation shaft defines at least three inflation lumens, each lumen being in fluid communication with a respective expandable balloon.

19. The system of claim 14 wherein the expandable balloons comprise an un-symmetric shape.

20. The system of claim 19 wherein the expandable balloons each define a flattened surface and a curved or arcuate surface.

21. The system of claim 14 wherein the expandable balloons comprise a spherical shape.

22. The system of claim 1 further comprising an expandable stent in the at least one support member configured to provide structural support to maintain the support member in an expanded configuration.

23. The system of claim 1 further comprising a coupling lumen positioned along a proximal end of the at least one support member, the coupling lumen having an expandable coupling mechanism positioned therein.

24. The system of claim 23 wherein the expandable coupling mechanism comprises a stent or crimp.

25. The system of claim 23 further comprising an outer catheter coupled to the at least one support member via the coupling mechanism within the coupling lumen.

26. The system of claim 25 further comprising an expandable release mechanism extending distally from the outer catheter within the coupling mechanism, wherein expansion of the release mechanism de-couples the coupling mechanism from the outer catheter.

27. A method of supporting a posterior mitral leaflet, comprising:
intravascularly advancing a delivery catheter into a left ventricle such that a distal portion of the delivery catheter is adjacent to an inferior surface of a posterior mitral leaflet in the patient body;
positioning at least one support member having at least one attractive element thereon with a first polarity adjacent to the inferior surface of the posterior mitral leaflet via the delivery catheter;
intravascularly advancing a guiding catheter into a coronary sinus such that a distal portion of the guiding catheter is adjacent to a mitral valve in a patient body;
positioning at least one anchoring element with a second polarity opposite to the first polarity within the coronary sinus via the guiding catheter such that the at least one support member and anchoring element are magnetically engaged with one another; and
expanding the at least one support member against the inferior surface of the posterior mitral leaflet such that prolapse of the leaflet is inhibited.

28. The method of claim 27 wherein intravascularly advancing a delivery catheter comprises advancing the delivery catheter until a magnetic element positioned on a distal tip on the delivery catheter is attracted to a corresponding magnetic element positioned on a distal tip of the guiding catheter.

29. The method of claim 27 wherein intravascularly advancing a delivery catheter further comprises anchoring the delivery catheter via an inflatable balloon at its distal tip.

30. The method of claim 27 wherein expanding comprises inflating one or more balloon members against the inferior surface of the posterior mitral leaflet.

31. The method of claim 30 further comprising expanding at least one stent member within each of the balloon members such that each stent maintains an expanded configuration of the balloon members.

32. The method of claim 27 wherein expanding comprising inflating at least three balloon members independently of one another against the inferior surface.

33. The method of claim 31 wherein expanding comprises adjusting an inflation of one or more balloon members.

34. The method of claim 27 further comprising releasing a coupling mechanism connecting the at least one support member to an outer catheter.

35. The method of claim 34 further comprising removing the outer catheter such that the at least one support member is magnetically retained against the inferior surface of the posterior mitral leaflet.

36. A method of supporting a posterior mitral leaflet, comprising:
positioning a ringed support member at least partially around a chordae tendineae supporting a mitral valve;
sliding the ringed support member along the chordae tendineae into a superior position against or adjacent to a posterior mitral leaflet during systole; and
sliding the ringed support member along the chordae tendineae into an inferior position towards a papillary muscle during diastole.

37. The method of claim 36 wherein sliding the ringed support member along the chordae tendineae into a superior position comprises urging the support member via tissue contractions and blood flow.
